SEQUENCE LISTING (10 sequences)

SEQ ID NO: 1 - DNA, 24 bp, synthetic construct
  source 1..24: mol_type = other DNA
ggcataagtgatcctcagacataa

SEQ ID NO: 2 - DNA, 19 bp, synthetic construct
  source 1..19: mol_type = other DNA
agttggcgcatcccataaa

SEQ ID NO: 3 - DNA, 21 bp, synthetic construct
  source 1..21: mol_type = other DNA
ggaacacatgaccgtgtatga

SEQ ID NO: 4 - DNA, 22 bp, synthetic construct
  source 1..22: mol_type = other DNA
tctctcgactctctgctatgaa

SEQ ID NO: 5 - DNA, 22 bp, synthetic construct
  source 1..22: mol_type = other DNA
agagggatcttggatggaaatg

SEQ ID NO: 6 - DNA, 21 bp, synthetic construct
  source 1..21: mol_type = other DNA
tgaggactttggtgtgttgag

SEQ ID NO: 7 - DNA, 27 bp, synthetic construct
  source 1..27: mol_type = other DNA
cctcaaacgagacacagacagacttag

SEQ ID NO: 8 - DNA, 22 bp, synthetic construct
  source 1..22: mol_type = other DNA
gatcatactcaccaagcctcac

SEQ ID NO: 9 - DNA, 20 bp, synthetic construct
  source 1..20: mol_type = other DNA
cagtacagccccaaaatggt

SEQ ID NO: 10 - DNA, 20 bp, synthetic construct
  source 1..20: mol_type = other DNA
caagggcatatccaacaaca

(19) United States
(12) Patent Application Publication
(10) Pub. No.: US 2026/0337682 A1
(43) Pub. Date: Oct. 29, 2026

(54) DIAGNOSIS AND TREATMENT OF ACUTE KIDNEY INJURY

(71) Applicant: The Johns Hopkins University, Baltimore, MD (US)

(72) Inventors: Chirag Parikh, Lutherville, MD (US); Yumeng Wen, Baltimore, MD (US)

(21) Appl. No.: 19/104,947

(22) PCT Filed: Aug. 18, 2023
(86) PCT No.: PCT/US2023/072477

§ 371 Date: Feb. 19, 2025

Related U.S. Application Data
(60) Provisional application No. 63/371,894, filed on Aug. 19, 2022.

(51) Int. Cl. C12Q 1/6883

(52) U.S. Cl. CPC C12Q 1/6883; C12Q 2600/118; C12Q 2600/158

(57) ABSTRACT

The present invention relates to the field of nephrology. More specifically, the present invention provides compositions and methods useful for the diagnosis and treatment of acute kidney injury. In a specific embodiment, a method for identifying a patient as having proximal tubular injury (PTI) comprises the step of detecting the amount of one or more of COL23A1, TGFB2, FSTL3, NLGN4X, ENPP6, PLG, P4HA2 and AFM in a biological sample obtained from the patient, wherein an increase in the amount of one or more of COL23A1, TGFB2, FSTL3, NLGN4X, ENPP6, PLG, P4HA2 and AFM relative to a reference identifies the patient as having PTI.

### CROSS-REFERENCE TO RELATED APPLICATIONS

This application claims the benefit of U.S. Provisional Application No. 63/371,894, filed Aug. 19, 2022, which is incorporated herein by reference in its entirety.

### GOVERNMENT SUPPORT CLAUSE

This invention was made with government support under grant no. DK114866 and grant no. HL085757, awarded by the National Institutes of Health. The government has certain rights in the invention.

### FIELD OF THE INVENTION

The present invention relates to the field of nephrology. More specifically, the present invention provides compositions and methods useful for the diagnosis and treatment of acute kidney injury.

### REFERENCE TO AN ELECTRONIC SEQUENCE LISTING

The text of the computer readable sequence listing filed herewith, titled “P17069-02”, created Aug. 8, 2023, having a file size of 9,812 bytes, is hereby incorporated by reference in its entirety.

### BACKGROUND OF THE INVENTION

Acute kidney injury (AKI) represents an acute decline in kidney function and is often caused by ischemia and toxic insults. It is common in hospitalized patients and is associated with significant morbidity and mortality. The AKI diagnosis and staging criteria across these definitions revolve around changes in serum creatinine (SCr), urine output, and requirement for kidney replacement therapy (KRT). However, change in SCr or urine output are not sensitive to detect early injury to the renal tubule, including the proximal tubule which is the primary target of injury in AKI. In addition, SCr may increase in various clinical scenarios of AKI, such as pre-renal azotemia, acute tubular injury (ATI), hepatorenal syndrome (HRS), and cardiorenal syndrome, which have diverse pathophysiological processes and require different management strategies. This unspecific rise in SCr during AKI makes both the diagnosis and patient management challenging.

### SUMMARY OF THE INVENTION

In mouse acute kidney injury (AKI), a subgroup of injured proximal tubule (PT) cells undergo maladaptive changes that prevent complete tissue recovery. However, evidence for PT maladaptation and its etiological relationship with complications of AKI are lacking in humans. The present inventors aimed to elucidate the transcriptomic landscape of PT maladaptation in humans, identify plasma proteomic signatures linked to the maladaptive process, and determine their associations with adverse renal outcomes. As described herein, the present inventors performed single-nucleus RNA sequencing of 120,985 nuclei in kidneys from 17 participants with AKI and 7 healthy controls from the Kidney Precision Medicine Project. The phenotype of maladaptive PT, characterized by dedifferentiation and enrichment in ferroptotic, necroptotic, proinflammatory, and profibrotic pathways, was widely present in participants with AKI of diverse etiologies. The present inventors measured the plasma proteome (SOMAscan) in 2 independent cohorts of cardiac surgery patients and a cohort of marathon runners, linked it to the transcriptomic signatures associated with maladaptive PT, and identified 9 proteins whose genes were specifically up- or downregulated by maladaptive PT. After cardiac surgery, both cohorts of patients had increases in TGFB2, COL23A1, and NLGN4X, and decreases in PLG, ENPP6, and PROC. Similar changes were observed in marathon runners with exercise-associated injury. The postoperative changes in these biomarkers were strongly associated with severe AKI in adults after cardiac surgery and progression to post-AKI kidney atrophy in mouse models of ischemia-reperfusion injury and toxic injury. The present inventors' results demonstrate the feasibility of a multiomics approach to discovering novel, noninvasive markers and associate PT maladaptation with adverse clinical outcomes.

In particular embodiments, the present invention utilizes eight plasma proteins for the diagnosis of proximal tubular injury. These proteins were identified via a multiomics approach, integrating plasma proteomics and kidney tissue transcriptomics from two large cohorts of patients. The present inventors show herein that the genes of these proteins were specifically upregulated by the injured proximal tubular cells in the kidney, and, in certain embodiments, the increase in the plasma levels of these proteins after cardiac surgery is associated with higher risk of developing severe AKI.

In more particular embodiments, the panel comprises seven plasma protein biomarkers: neuregulin-4 X linked (NLGN4X), collagen type XXIII α 1 chain (COL23A1) transforming growth factor β-2 (TGFB2)), ectonucleotide pyrophosphatase/phosphodiesterase 6 (ENPP6), plasminogen (PLG) and protein C (PROC). In further embodiments, the panel further comprises CD200, prolyl 4-hydroxylase (P4HA2) and/or afamin (AFM).

In certain embodiments, the present invention can be used for early detection of drug toxicity. Drug toxicity is a common cause of acute tubular injury and AKI. Drugs commonly associated with tubular injury includes antibiotics (vancomycin, piperacillin-tazobactam and aminoglycosides), antivirals (acyclovir, tenofovir), antifungal (colistin), lithium, nonsteroidal anti-inflammatory drugs and kidney injury drugs (methotrexate, cisplatin and ifosfamide). SCr is often unchanged until tubular injury is severe, resulting a delay in cessation of these nephrotoxic agents and worsening kidney function. The present invention can provide early detection of drug induced proximal tubular injury before SCr starts to increase and the clinical diagnosis of AKI is made. The present invention can be used to routinely monitor for toxicity, clinicians may promptly stop these nephrotoxic agents when proximal tubular injury occurs and prevent further injury.

In other embodiments, the present invention can be used in the management of cardiorenal syndrome and hepatorenal syndrome. The diagnosis and management of AKI in patients with decompensated cirrhosis largely depends on its etiologies and pathophysiological processes. Hepatorenal syndrome (HRS), pre-renal azotemia, and ATI are the most common etiologies in these patients. Each requires a completely different management strategy. HRS, marked by splenic vasodilatation and decreased renal perfusion through neurohormonal activation, may be reversible after administration of vasoconstrictive therapy. Pre-renal azotemia requires volume expansion with either albumin or crystalloid. ATI could potentially progress and often warrants evaluation of simultaneous liver and kidney transplantation. The proposed biomarker panel may be used to evaluate ATI in patients with decompensated cirrhosis. Lack of change in these biomarkers may ensure the health of tubules, prompt the use of volume expansion and vasoconstrictive therapy, and may indicate recovery potentials after liver transplantation. On the other hand, a marked increase in these biomarkers may indicate severe injury to the proximal tubules and the evaluation of simultaneous liver kidney transplantation should be pursued.

Similarly, the management of AKI in patients with decompensated heart failure is also challenging, as SCr based AKI diagnosis criteria does not differentiate kidney function fluctuation from hemodynamic changes from true intrinsic kidney injury. For patients with decompensated heart failure, aggressive diuresis is often necessary to relieve renal congestion and restore hemodynamics. However, diuresis was often held when SCr rises from hemoconcentration without evidence of true intrinsic renal injury, resulting in incomplete decongestion, higher risk of readmission and worse mortality outcome. The present invention can be used to rule out the presence of intrinsic tubular injury when SCr rises during decongestion and facilitate aggressive fluid removal. Conversely, the increase in these biomarkers may indicate the presence of proximal tubular injury and alert physicians that other reasons for AKI should be evaluated.

In further embodiments, the present invention can be used for risk stratification for AKI transitioning to chronic kidney disease. In recent years, much work has focused on the long-term morbidity and mortality of AK. Patients with severe (KDIGO stage 2-3) AKI have two-fold higher risk of developing CKD comparing to those with mild AKI (Hsu et al JAMA Intern Med, 2020), and 5-8 fold higher risk of CKD comparing to those without AKI (Coca S G et al, Kidney Int. 2012). The proposed panel of biomarkers are strongly associated with developing severe AKI, and thus may also be associated with developing incident CKD or experiencing CKD progression after severe AKI. These biomarkers could be used for risk stratification and for selection of patients that are at high risk of developing CKD after AKI. Timely initiation of nephroprotective medications, such as angiotensin converting enzyme inhibitors, angiotensin receptor blockers and sodium glucose cotransporter inhibitors, may help prevent the development and progression of CKD after hospitalization with AKI.

In other embodiments, the present invention can be used for the detection of drug toxicity in preclinical rodent toxicity studies. Several urine biomarkers, such as urine albumin, beta2-microglobulin, clusterin, cystatin-C, KIM-1 were previously approved by the FDA for preclinical use to indicate renal injury in rat. The proposed panel of biomarkers may be used for evaluation of nephrotoxicity as an addition to standard laboratory data used in rodent safety assessment studies. They will provide additional information to SCr and blood urea nitrogen, and correlates with histological renal tubular injury. The increase in COL23A1, TGFB2, and NLGN4X, and the decrease in ENPP6, PLG, P4HA2 and AFM may indicate early injury to the proximal tubule in rodents and indicate significant nephrotoxicity.

Accordingly, in one aspect, the present invention provides methods for detecting acute tubular injury (ATI). In a specific embodiment, the method comprises the steps of (a) detecting increased expression of neuregulin-4 X linked (NLGN4X), collagen type XXIII α 1 chain (COL23A1) and transforming growth factor β-2 (TGFB2) relative to a reference in a sample obtained from a patient; and (b) detecting decreased expression of ectonucleotide pyrophosphatase/phosphodiesterase 6 (ENPP6), plasminogen (PLG) and protein C (PROC) in the patient sample. In another specific embodiment, step (a) further comprises detecting expression of CD200. In yet another embodiment, step (b) further comprising detecting expression of prolyl 4-hydroxylase (P4HA2) and/or afamin (AFM). In certain embodiments, ATI comprises proximal tubular injury (PTI). In any of the embodiments described herein, the biological sample can be, but is not limited to, plasma or urine.

The present invention also provides methods for identifying a patient as likely to develop acute kidney injury (AKI) comprising the steps of: (a) detecting increased expression levels of NLGN4X, COL23A1, and TFGB2 relative to a reference in a sample obtained from the patient; and (b) detecting decreased expression levels of ENPP6, PLG and PROC relative to a reference in the patient sample, thereby detecting ATI in the patient which is likely to develop into AKI. In another specific embodiment, step (a) further comprises detecting expression of CD200. In yet another embodiment, step (b) further comprising detecting expression of P4HA2 and/or AFM. In certain embodiments, ATI comprises PTI.

In particular embodiments, the patient identified in step (b) is treated for AKI. The AKI treatment can comprise, but is not limited to, one or more of renal replacement therapy, angiotensin-converting-enzyme inhibitors, Ang II type I (ATI) blockers, corticosteroids, immunosuppressants, artemisinin and derivatives thereof, drag-reducing polymers, endothelin subtype A receptor antagonists (pro)renin receptor antagonists and 5-HT₃-targeting drugs.

In a specific embodiment, a method comprises the step of detecting the amount of NLGN4X, COL23A1, TGFB2, ENPP6, PLG, PROC and optionally one or more of CD200, P4HA2 and AFM in a biological sample obtained from a patient.

In another embodiment, a method for identifying a patient as having PTI comprises the step of measuring the level of NLGN4X, COL23A1, TGFB2, ENPP6, PLG, PROC and optionally one or more of CD200, P4HA2 and AFM in a biological sample obtained from the patient, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference identifies the patient as having PTI.

In yet another embodiment, the present invention provides a method of identifying a cardiac surgery patient as having a high risk of developing severe AKI comprising the step of measuring the level of NLGN4X, COL23A1, TGFB2, ENPP6, PLG, PROC and optionally one or more of CD200, P4HA2 and AFM in a biological sample obtained from the patient, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference identifies the patient as having a high risk of developing severe AKI.

In a further embodiment, a method for detecting drug-induced PTI comprises the step of measuring the level of NLGN4X, COL23A1, TGFB2, ENPP6, PLG, PROC and optionally one or more of CD200, P4HA2 and AFM in a biological sample obtained from the patient, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference identifies the patient as having drug-induced PTI. In another embodiment, the method further comprises the step of ceasing administration of the drug. In yet another embodiment, the serum creatinine (SCr) level of the patient has not increased relative to a control and a diagnosis of AKI has not been made.

The present invention also provides methods for monitoring kidney injury in a patient with decompensated cirrhosis. In a specific embodiment, the method comprises the step of measuring the level of NLGN4X, COL23A1, TGFB2, ENPP6, PLG, PROC and optionally one or more of CD200, P4HA2 and AFM in a biological sample obtained from the patient, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference identifies the patient as having PTI, or (c) no change in the amount of one or more of NLGN4X, COL23A1, TGFB2, ENPP6, PLG, PROC and optionally one or more of CD200, P4HA2 and AFM relative to a reference identifies the patient not having PTI. In certain embodiments, a patient identified as having PTI is given a liver or kidney transplantation. In other embodiments, a patient identified in step (c) is administered volume expansion therapy and/or vasoconstrictive therapy.

In another specific embodiment, a method for managing AKI in a patient with decompensated heart failure comprises the step of measuring the level of NLGN4X, COL23A1, TGFB2, ENPP6, PLG, PROC and optionally one or more of CD200, P4HA2 and AFM in a biological sample obtained from the patient, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference identifies the patient as having PTI, or (c) no change in the amount of one or more of NLGN4X, COL23A1, TGFB2, ENPP6, PLG, PROC and optionally one or more of CD200, P4HA2 and AFM relative to a reference identifies the patient not having PTI. In yet another specific embodiment, a patient identified as having PTI is administered diuresis to relieve renal congestion and restore hemodynamics. In other embodiments, a patient identified in step (c) is treated for AKI, which can comprise one or more of renal replacement therapy, angiotensin-converting-enzyme inhibitors, Ang II type I (ATI) blockers, corticosteroids, immunosuppressants, artemisinin and derivatives thereof, drag-reducing polymers, endothelin subtype A receptor antagonists (pro)renin receptor antagonists and 5-HT₃-targeting drugs.

The present invention also provides methods for risk stratification for AKI transitioning to chronic kidney disease (CKD) comprising the step of measuring the level of NLGN4X, COL23A1, TGFB2, ENPP6, PLG, PROC and optionally one or more of CD200, P4HA2 and AFM in a biological sample obtained from the patient, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference identifies the patient as developing incident CKD or experiencing CKD progression after severe AKI. In particular embodiments, the method can further comprise the step of administering to the patient developing incident CKD or experiencing CKD progression after severe AKI one or more of angiotensin converting enzyme inhibitors, angiotensin receptor blockers and sodium glucose cotransporter inhibitors.

In yet another embodiment, the present invention provides a method for evaluating nephrotoxicity in a preclinical rodent toxicity study comprising the step of measuring the level of NLGN4X, COL23A1, TGFB2, ENPP6, PLG, PROC and optionally one or more of CD200, P4HA2 and AFM in a biological sample obtained from the rodent who has been administered a drug, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference indicates early injury to the proximal tubules and significant nephrotoxicity.

### BRIEF DESCRIPTION OF THE FIGURES

FIG. 1A-1H. SnRNA-seq analysis of 17 participants with AKI and 7 healthy references from the KPMP cohort identified PT cells at different states of health. (FIG. 1A) UMAP of 120,985 kidney epithelium, stroma, and immune cell nuclei. (FIG. 1B) Dot plot of canonical marker gene expression of major kidney cell types. (FIG. 1C) UMAP of PT subclusters. (FIG. 1D) Dot plot of marker gene expression of PT subclusters demonstrated that PT cells in healthy and diseased states in the overall biopsy cohort. (FIG. 1E) Bar plot of PT subcluster composition in 17 AKI participants and 7 healthy references. (FIG. 1F-1H) Dot plot displaying enriched gene ontology pathways (FIG. 1F), and genes involved in ferroptosis pathways (FIG. 1G) and necroptosis pathways (FIG. 1H) among PT subclusters.

Abbreviations: AKI, acute kidney injury; CD, collecting duct; CNT, connecting tubule; DC, dendritic cell; DCT, distal convoluted tubule; EC, endothelial cell; Fib, fibroblast; Glom, glomerulus; ICA, intercalated cell of collecting duct type A; ICB, intercalated cell of collecting duct type B; Mac, macrophage; MD, macula densa; Mes, mesangial cell; Mono, monocyte; PC, principal cell of collecting duct; Per, pericyte; Pod, podocyte; PT, proximal tubule; TAL, thick ascending limb of loop of Henle; TL: thin limb of loop of Henle.

FIG. 2A-2D. Gene regulatory network analysis of PT subclusters in 17 participants with AKI demonstrated distinct regulatory networks in PT cells at different states of health. (FIG. 2A) Heatmap depicting average regulon enrichment in each PT subcluster. (FIG. 2B) Average expression of selected regulons enriched in each PT subcluster. (FIG. 2C) Heatmap demonstrating unsupervised clustering of PT subclusters by the top 10 regulons from each subcluster, revealing significant differences in regulon activity in PT cells in healthy and diseased states. (FIG. 2D) Louvain clustering of the top 10% of transcription factor-target gene pairs demonstrates clusters of transcription factors (nodes depicted by colors) forming coregulatory networks.

FIG. 3A-3B. Integration of the kidney tissue transcriptome (snRNA-seq) in participants with AKI and plasma proteome (SOMAscan) in cardiac surgery patients identified biomarkers of PT maladaptation (FIG. 3A) and PT cells at healthy states (FIG. 3B). Abbreviations: AKI, acute kidney injury; PT, proximal tubule; snRNA-seq, single-nucleus RNA sequencing.

FIG. 4A-4L. Kidney gene expression of biomarkers of PT maladaptation and PT cells at healthy states in 17 participants with AK and in mouse models of AK1. (FIG. 4A) Tissue gene expression of biomarkers in 17 participants with AKI from the KPMP cohort at single-nucleus resolution. (FIG. 4B-4F) Wild-type mice were subjected to unilateral IRI (atrophy model) or IRI with contralateral nephrectomy (repair model) (35). Quantitative RT-PCR analysis was performed on whole kidney RNA harvested 0 (healthy control), 1, 7, 14, and 30 days after injury; n=9-10 kidneys/timepoint/model. *P<0.05; **P<0.01; ****P<0.0001 at the indicated timepoints (by Sidak post-tests). (FIG. 4G-4L) Wild-type mice were subjected to intraperitoneal injection of 5 mg/kg aristolochic acid (n=14) or vehicle (n=4). Blood samples were collected at day 0, 3, 7, 10, 14, and 21 for blood urea nitrogen (BUN) measurements. Quantitative RT-PCR analysis was performed on whole kidney RNA harvested on days 0 (baseline), 7 (AKI phase) and 21 (CKD phase) after aristolochic acid injection, and day 21 (controls) after vehicle injection. *P<0.05, ***P<0.001, and ****P<0.0001 comparing gene expression to baseline (by Student t tests).

Abbreviations: AAN, aristolochic acid nephropathy; AFM, afamin; AKI, acute kidney injury; BUN, blood urea nitrogen; COL23A1, collagen type XXIII α 1 chain; CD, collecting duct; CKD, chronic kidney disease; CNT, connecting tubule; DC, dendritic cell; DCT, distal convoluted tubule; EC, endothelial cell; ENPP6, ectonucleotide pyrophosphatase/phosphodiesterase 6; Fib, fibroblast; Glom, glomerulus; ICA, intercalated cell of collecting duct type A; ICB, intercalated cell of collecting duct type B; IRI, ischemia-reperfusion injury; Mac, macrophage; MD, macula densa; Mes, mesangial cell; Mono, monocyte; NLGN4X, neuroligin-4 X linked; P4HA2, prolyl 4-hydroxylase; PC, principal cell of collecting duct; Per, pericyte; PLG, plasminogen; Pod, podocyte; PROC, protein C; PT, proximal tubule; RT-PCR, reverse transcription polymerase chain reaction; TAL, thick ascending limb of loop of Henle; TL: thin limb of loop of Henle; TGFB2, transforming growth factor β-2.

FIG. 5. Tissue gene expression of 39 candidate biomarkers of PT maladaptation identified from the integrated workflow in KPMP participants with AKI.

FIG. 6. Tissue gene expression of 38 candidate biomarkers of PT cells at healthy states identified from the integrated workflow in KPMP participants with AKI.

FIG. 7A-7C. Tissue gene expression of biomarkers of PT maladaptation and PT cells at healthy states in kidney biopsy tissues from 3 recently enrolled KPMP participants with AKI. (FIG. 7A) Marker gene expression of major kidney cell types in kidney biopsy tissues from 3 recently enrolled KPMP participants with AKI. (FIG. 7B) Marker gene expression of predicted PT subclusters using PT cells from 17 KPMP participants with AKI in the biomarker discovery phase. (FIG. 7C) Gene expression of biomarkers of PT maladaptation and PT cells at healthy states in predicted PT subclusters and other major kidney cell types.

FIG. 8A-8B. Tissue gene expression of biomarkers of PT maladaptation and PT cells at healthy states in kidney autopsy tissues from an independent cohort of critically ill patients with AKI. (FIG. 8A) Marker gene expression of PT subclusters classified using approaches described by Hinze et al (33). (FIG. 8B) Gene expression of biomarkers of PT maladaptation and PT cells at healthy states in PT subclusters and other kidney cell types.

FIG. 9. Correlation between gene expression of biomarkers of PT maladaptation and PT cells at healthy states with fibrosis markers in the recovery phase of AKI from mouse models of IRI.

FIG. 10. Gene expression of biomarkers of PT maladaptation and PT cells at healthy states in a publicly available snRNA-seq dataset of an AAN model. SnRNA-seq data published by Lu et al (37) was downloaded from the ArrayExpress database (accession code No. E-MTAB-9390). Gene expression of biomarkers on day 0 and day 28 after aristolochic acid injection (4 doses of 2.5 mg/kg over 2 weeks) is visualized after converting snRNA-seq count data to counts per million.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

The present invention integrates transcriptomics in the kidney tissue with proteomics in peripheral blood and identifies eight protein biomarkers of proximal tubule injury. Specifically, the present inventors developed a work-flow that identifies unique genes that are expressed by injured proximal tubule cells from single nuclei RNA sequencing analysis. The present inventors then integrated with blood proteomic profiles in patients who developed acute kidney injury (AKI), and identified eight protein biomarkers that can be measured in peripheral blood and reflected the severity of proximal tubular injury. These biomarkers include FSTL3, NLGN4X, COL23A1, TGFB2, P4HA2, ENPP6, PLG and AFM. These biomarkers of proximal tubular injury are associated with the development of moderate to severe acute kidney injury (AKI) after cardiac surgery. They may be used as markers for the assessment of proximal tubule health in patients at high risk for developing AKI and providing prognosis in patients with existing AKI. These proteins may also be mechanistically involved in injury and repair processes for AKI and may have therapeutic value.

In further embodiments, the present invention provides risk stratification in the post-operative setting in predicting the development of moderate to severe AKI after cardiac surgery. It also has potential to provide prognosis assessment for kidney function recovery, and long-term kidney outcome, such as developing chronic kidney disease and end stage kidney disease in future studies. Measurement platforms using standardized immunoassay can be developed based on this invention for future clinical application.

### I. DEFINITIONS

As used herein, a “subject”, “patient” or “individual” is a human. A subject can be one who has been previously diagnosed with or identified as suffering from or having a condition, disease, or disorder in need of treatment (e.g., kidney injury) or one or more complications related to the condition, disease, or disorder, and optionally, have already undergone treatment for the condition, disease, disorder, or the one or more complications related to the condition, disease, or disorder. Alternatively, a subject can also be one who has not been previously diagnosed as having a condition, disease, or disorder or one or more complications related to the condition, disease, or disorder. For example, a subject can be one who exhibits one or more risk factors for a condition, disease, or disorder, or one or more complications related to the condition, disease, or disorder, or a subject who does not exhibit risk factors. A “subject in need” of treatment for a particular condition, disease, or disorder can be a subject suspected of having that condition, disease, or disorder, diagnosed as having that condition, disease, or disorder, already treated or being treated for that condition, disease, or disorder, not treated for that condition, disease, or disorder, or at risk of developing that condition, disease, or disorder.

In some embodiments, the subject is selected from the group consisting of a subject suspected of having a disease, a subject that has a disease, a subject diagnosed with a disease, a subject that has been treated for a disease, a subject that is being treated for a disease, and a subject that is at risk of developing a disease.

By “at risk of” is intended to mean at increased risk of, compared to a normal subject, or compared to a control group, e.g., a patient population. Thus, a subject carrying a particular marker may have an increased risk for a specific condition, disease or disorder, and be identified as needing further testing. “Increased risk” or “elevated risk” mean any statistically significant increase in the probability, e.g., that the subject has the disorder. The risk is increased by at least 10%, at least 20%, and even at least 50% over the control group with which the comparison is being made. In certain embodiments, a subject can be at risk of developing kidney injury.

“Sample” is used herein in its broadest sense. The term “biological sample” as used herein denotes a sample taken or isolated from a biological organism. A sample or biological sample may comprise a bodily fluid including blood, serum, plasma, tears, aqueous and vitreous humor, spinal fluid; a soluble fraction of a cell or tissue preparation, or media in which cells were grown; or membrane isolated or extracted from a cell or tissue; polypeptides, or peptides in solution or bound to a substrate; a cell; a tissue, a tissue print, a fingerprint, skin or hair; fragments and derivatives thereof. Non-limiting examples of samples or biological samples include cheek swab; mucus; whole blood, blood, serum; plasma; urine; saliva, semen; lymph; fecal extract; sputum; other body fluid or biofluid; cell sample; and tissue sample etc. The term also includes a mixture of the above-mentioned samples or biological samples. The term “sample” also includes untreated or pretreated (or pre-processed) biological samples. In some embodiments, a sample or biological sample can comprise one or more cells from the subject. Subject samples or biological samples usually comprise derivatives of blood products, including blood, plasma and serum. In some embodiments, the sample is a biological sample. In some embodiments, the sample is blood. In some embodiments, the sample is peripheral blood.

The terms “body fluid” or “bodily fluids” are liquids originating from inside the bodies of organisms. Bodily fluids include amniotic fluid, aqueous humour, vitreous humour, bile, blood (e.g., serum), breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, sputum, synovial fluid, sweat, tears, urine, vaginal secretion, and vomit. Extracellular bodily fluids include intravascular fluid (blood plasma), interstitial fluids, lymphatic fluid and transcellular fluid. “Biological sample” also includes a mixture of the above-mentioned body fluids. “Biological samples” may be untreated or pretreated (or pre-processed) biological samples. In particular embodiments, body fluid means urine.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD Mitra microsampling device for accurate dried blood sampling (Neoteryx), HemaSpot™-HF Blood Collection Device, a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMS^{IM}) samples can be stored and mailed, and an assay can be performed remotely.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, -carboxyglutamate, and O-phosphoserine. Amino acid analogs refers to compounds that have the same basic chemical structure as a naturally occurring amino acid, i.e., an carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function s in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “peptide” as used herein refers to any compound containing at least two amino acid residues joined by an amide bond formed from the carboxyl group of one amino acid residue and the amino group of the adjacent amino acid residue. In some embodiments, peptide refers to a polymer of amino acid residues typically ranging in length from 2 to about 30, or to about 40, or to about 50, or to about 60, or to about 70 residues. In certain embodiments the peptide ranges in length from about 2, 3, 4, 5, 7, 9, 10, or 11 residues to about 60, 50, 45, 40, 45, 30, 25, 20, or 15 residues. In certain embodiments the peptide ranges in length from about 8, 9, 10, 11, or 12 residues to about 15, 20 or 25 residues. In some embodiments, the peptide ranges in length from 2 to about 12 residues, or 2 to about 20 residues, or 2 to about 30 residues, or 2 to about 40 residues, or 2 to about 50 residues, or 2 to about 60 residues, or 2 to about 70 residues. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues are an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an a-ester, a f3-ester, a thioamide, phosphonamide, carbamate, hydroxylate, and the like (see, e.g., Spatola, (1983) Chem. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn et al., (1990) pp. 969-970 in Proc. 11th American Peptide Symposium, ESCOM Science Publishers, The Netherlands, and the like)).

A protein refers to any of a class of nitrogenous organic compounds that comprise large molecules composed of one or more long chains of amino acids and are an essential part of all living organisms. A protein may contain various modifications to the amino acid structure such as disulfide bond formation, phosphorylations and glycosylations. A linear chain of amino acid residues may be called a “polypeptide,” A protein contains at least one polypeptide. Short polypeptides, e.g., containing less than 20-30 residues, are sometimes referred to as “peptides.”

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy-chain variable region.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

The term “threshold” as used herein refers to the magnitude or intensity that must be exceeded for a certain reaction, phenomenon, result, or condition to occur or be considered relevant. The relevance can depend on context, e.g., it may refer to a positive, reactive or statistically significant relevance.

By “binding assay” is meant a biochemical assay wherein the biomarkers are detected by binding to an agent, such as an antibody, through which the detection process is carried out. The detection process may involve fluorescent or radioactive labels, and the like. The assay may involve immobilization of the biomarker, or may take place in solution.

“Immunoassay” is an assay that uses an antibody to specifically bind an antigen (e.g., a marker). The immunoassay is characterized by the use of specific binding properties of a particular antibody to isolate, target, and/or quantify the antigen. Non-limiting examples of immunoassays include ELISA (enzyme-linked immunosorbent assay), immunoprecipitation, SISCAPA (stable isotope standards and capture by anti-peptide antibodies), Western blot, etc.

“Diagnostic” means identifying the presence or nature of a pathologic condition, disease, or disorder and includes identifying patients who are at risk of developing a specific condition, disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, a disease, or a disorder, it suffices if the method provides a positive indication that aids in diagnosis.

The term “statistically significant” or “significantly” refers to statistical evidence that there is a difference. It is defined as the probability of making a decision to reject the null hypothesis when the null hypothesis is actually true. The decision is often made using the p-value.

As used herein, the term “sensitivity” refers to the ability of a method to detect or identify the presence of a disease or condition in a subject. For example, when used in reference to any of the variety of methods described herein that can detect the presence of proximal tubular injury (PTI) in a subject (e.g., acute kidney injury), a high sensitivity means that the method correctly identifies the presence of PTI in the subject a large percentage of the time. For example, a method described herein that correctly detects PTI in a subject 95% of the time the method is performed is said to have a sensitivity of 95%. In particular embodiments, a method described herein that can detect PTI in a subject provides a sensitivity of at least 70% (e.g., about 70%, about 72%, about 75%, about 80%, about 85%, about 90%, about 91%, about 92%, about 93%, about 94%, about 95%, about 96%, about 97%, about 98%, about 99%, about 99.5%, or about 100%). In certain embodiments, methods provided herein that include detecting the presence of one or more members of two or more classes of biomarkers (e.g., nucleic acid biomarkers and/or protein biomarkers) provide a higher sensitivity than methods that include detecting the presence of one or more members of only one class of biomarkers.

As used herein, the term “specificity” refers to the ability of a method to detect the presence of a disease or condition in a subject (e.g., the specificity of a method can be described as the ability of the method to identify the true positive over true negative rate in a subject and/or to distinguish a truly occurring sequence variant from a sequencing artifact or other closely related sequences). For example, when used in reference to any of the variety of methods described herein that can detect the presence of kidney injury (e.g., acute kidney injury) in a subject, a high specificity means that the method correctly identifies the absence of kidney injury in the subject a large percentage of the time (e.g., the method does not incorrectly identify the presence of kidney injury in the subject a large percentage of the time). In some embodiments, a method described herein that can detect the absence of kidney injury (e.g., normal) in a subject provides a specificity of at least 80% (e.g., at least 80%, 85%, 90%, 91%, 92%, 93%, 94%, 95%, 96%, 97%, 98%, 99%, 99.5%, or higher). A method having high specificity results in minimal or no false positive results (e.g., as compared to other methods). False positive results can arise from any source. In some embodiments, methods provided herein that include detecting the presence of one or more members of two or more classes of biomarkers (e.g., nucleic acid biomarkers and/or protein biomarkers) provide a higher specificity than methods that include detecting the presence of one or more members of only one class of biomarkers.

The terms “detection”, “detecting” and the like, may be used in the context of detecting biomarkers, detecting peptides, detecting proteins, or of detecting a condition, detecting a disease or a disorder (e.g., when positive assay results are obtained). In the latter context, “detecting” and “diagnosing” are considered synonymous when mere detection indicates the diagnosis. The term is also used synonymously with the term “measuring.”

The terms “marker” or “biomarker” are used interchangeably herein, and in the context of the present invention refer to a protein or peptide (for example, protein or peptide associated with kidney injury as described herein) is differentially present in a sample taken from patients having a specific disease or disorder as compared to a control value, the control value consisting of, for example average or mean values in comparable samples taken from control subjects (e.g., a person with a negative diagnosis, normal or healthy subject). Biomarkers may be determined as specific peptides or proteins which may be detected by, for example, antibodies or mass spectroscopy. In some applications, for example, a mass spectroscopy or other profile of multiple antibodies may be used to determine multiple biomarkers, and differences between individual biomarkers and/or the partial or complete profile may be used for diagnosis. In some embodiments, the biomarkers may be detected by antibodies, mass spectrometry, or combinations thereof.

A “test amount” of a marker refers to an amount of a marker present in a sample being tested. A test amount can be either in absolute amount (e.g., g/mL) or a relative amount (e.g., relative intensity of signals).

A “diagnostic amount” of a marker refers to an amount of a marker in a subject's sample that is consistent with a diagnosis of a particular disease or disorder. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person who does not suffer from the disease or disorder sought to be diagnosed, A control amount can be either in absolute amount (e.g., μg/ml) or a relative amount (e.g., relative intensity of signals).

The term “differentially present” or “change in level” refers to differences in the quantity and/or the frequency of a marker present in a sample taken from patients having a specific disease or condition as compared to a control subject. For example, a marker can be present at an elevated level or at a decreased level in samples of patients with the disease or condition compared to a control value (e.g., determined from samples of control subjects). Alternatively, a marker can be detected at a higher frequency or at a lower frequency in samples of patients compared to samples of control subjects. A marker can be differentially present in terms of quantity, frequency or both as well as a ratio of differences between two or more specific modified amino acid residues and/or the protein itself. In particular embodiments, a marker can be differentially present in patients having PTI as compared to a control subject including patients having no PTI.

A marker, compound, composition or substance is differentially present in a sample if the amount of the marker, compound, composition or substance in the sample (e.g., a patient having acute kidney injury) is statistically significantly different from the amount of the marker, compound, composition or substance in another sample (e.g., a patient having acute kidney injury or no kidney injury), or from a control value (e.g., an index or value representative of acute kidney injury or no kidney injury). For example, a marker is differentially present if it is present at least about 50%, at least about 60%, at least about 70%, at least about 80%, at least about 90%, at least about 100%, at least about 110%, at least about 120%, at least about 130%, at least about 150%, at least about 180%, at least about 200%, at least about 300%, at least about 500%, at least about 700%, at least about 900%, or at least about 1000% greater or less than it is presence in the other sample (e.g., control), or if it is detectable in one sample and not detectable in the other.

Alternatively, or additionally, a marker, compound, composition or substance is differentially present between samples if the frequency of detecting the marker, etc. in samples of patients suffering from a particular disease or condition, is statistically significantly higher or lower than in the control samples or control values obtained from controls such as a subject having PTI, acute kidney injury, and the like, or otherwise healthy individuals. For example, a biomarker is differentially present between the two sets of samples if it is detected at least about 10%, at least about 20%, at least about 30%, at least about 40%, at least about 50%, at least about 60%, at least about 70%, at least about 80%, at least about 90%, or at least about 100% more frequently or less frequently observed in one set of samples (e.g., a patient having PTI) than the other set of samples (e.g., a patient having PTI or no PTI). These exemplary values notwithstanding, it is expected that a skilled practitioner can determine cut-off points, etc., that represent a statistically significant difference to determine whether the marker is differentially present.

The term “one or more of” refers to combinations of various biomarkers. The term encompasses 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 36, 37, 38, 39, 40 . . . N, where “N” is the total number of biomarker proteins in the particular embodiment. The term also encompasses, and is interchangeably used with, at least 1, at least 2, at least 3, at least 4, at least 5, at least 6, at least 7, at least 8, at least 9, at least 10, at least 11, at least 12, at least 13, at least 15, 16, 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 . . . N. It is understood that the recitation of biomarkers herein includes the phrase “one or more of” the biomarkers and, in particular, includes the “at least 1, at least 2, at least 3” and so forth language in each recited embodiment of a biomarker panel.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, flow cytometry, or direct analysis by mass spectrometry of intact protein or peptides. In some embodiments, the detectable moiety is a stable isotope. In some embodiments, the stable isotope is selected from the group consisting of ¹⁵N, ¹³C, ¹⁸O and ²H.

As used herein, the terms “treat”, “treatment”, “treating”, or “amelioration” when used in reference to a disease, disorder or medical condition, refer to both therapeutic treatment and prophylactic or preventative measures, wherein the object is to reverse, alleviate, ameliorate, inhibit, lessen, slow down or stop the progression or severity of a symptom, a condition, a disease, or a disorder. The term “treating” includes reducing or alleviating at least one adverse effect or symptom of a condition, a disease, or a disorder. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition, disease, or disorder even if the treatment is ultimately unsuccessful. Those in need of treatment include those already with the condition, disease, or disorder as well as those prone to have the condition, disease, or disorder or those in whom the condition, disease, or disorder is to be prevented.

Non-limiting examples of treatments or therapeutic treatments include pharmacological or biological therapies and/or interventional surgical treatments.

The term “preventative treatment” means maintaining or improving a healthy state or non-diseased state of a healthy subject or subject that does not have a disease. The term “preventative treatment” or “health surveillance” also means to prevent or to slow the appearance of symptoms associated with a condition, disease, or disorder. The term “preventative treatment” also means to prevent or slow a subject from obtaining a condition, disease, or disorder. In particular embodiments, the condition is PTI.

As used herein, the term “administering,” refers to the placement an agent or a treatment as disclosed herein into a subject by a method or route which results in at least partial localization of the agent or treatment at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrastemai, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition.

### II. DETECTION/MEASUREMENT OF PROTEIN MARKERS

In specific embodiments, the proteins of the present invention can be detected and/or measured by immunoassay. Immunoassay requires biospecific capture reagents/binding agent, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art. Biospecific capture reagents useful in an immunoassay can also include lectins. In other embodiments, the biospecific capture reagents bind the specific biomarker and not similar forms thereof.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

In certain embodiments, the expression levels of the protein biomarkers employed herein are quantified by immunoassay, such as enzyme-linked immunoassay (ELISA) technology. In specific embodiments, the levels of expression of the biomarkers are determined by contacting the biological sample with antibodies, or antigen binding fragments thereof, that selectively bind to the biomarker; and detecting binding of the antibodies, or antigen binding fragments thereof, to the biomarkers. In certain embodiments, the binding agents employed in the disclosed methods and compositions are labeled with a detectable moiety. In other embodiments, a binding agent and a detection agent are used, in which the detection agent is labeled with a detectable moiety. For ease of reference, the term antibody is used in describing binding agents or capture molecules. However, it is understood that reference to an antibody in the context of describing an exemplary binding agent in the methods of the present invention also includes reference to other binding agents including, but not limited to lectins.

For example, the level of a biomarker in a sample can be assayed by contacting the biological sample with an antibody, or antigen binding fragment thereof, that selectively binds to the target protein (referred to as a capture molecule or antibody or a binding agent), and detecting the binding of the antibody, or antigen-binding fragment thereof, to the protein. The detection can be performed using a second antibody to bind to the capture antibody complexed with its target biomarker. A target biomarker can be an entire protein, or a variant or modified form thereof. Kits for the detection of proteins as described herein can include pre-coated strip/plates, biotinylated secondary antibody, standards, controls, buffers, streptavidin-horse radish peroxidase (HRP), tetramethyl benzidine (TMB), stop reagents, and detailed instructions for carrying out the tests including performing standards.

The present disclosure also provides methods for detecting protein in a sample obtained from a subject, wherein the levels of expression of the proteins in a biological sample are determined simultaneously. For example, in one embodiment, methods are provided that comprise: (a) contacting a biological sample obtained from the subject with a plurality of binding agents that each selectively bind to one or more biomarker proteins for a period of time sufficient to form binding agent-biomarker complexes; and (b) detecting binding of the binding agents to the one or more biomarker proteins. In further embodiments, detection thereby determines the levels of expression of the biomarkers in the biological sample; and the method can further comprise (c) comparing the levels of expression of the one or more biomarker proteins in the biological sample with predetermined threshold values, wherein levels of expression of at least one of the biomarker proteins above or below the predetermined threshold values indicates, for example, the subject has kidney injury, the severity of kidney injury, and/or is/will be responsive to kidney injury therapy. Such embodiments can assist in identifying whether a subject has, for example, AKI versus normal. Examples of binding agents that can be effectively employed in such methods include, but are not limited to, antibodies or antigen-binding fragments thereof, aptamers, lectins and the like.

Although antibodies are useful because of their extensive characterization, any other suitable agent (e.g., a peptide, an aptamer, or a small organic molecule) that specifically binds a biomarker of the present invention is optionally used in place of the antibody in the above-described immunoassays. For example, an aptamer that specifically binds a biomarker and/or one or more of its breakdown products might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

In specific embodiments, the assay performed on the biological sample can comprise contacting the biological sample with one or more capture agents (e.g., antibodies, lectins, peptides, aptamer, etc., combinations thereof) to form a biomarker:capture agent complex. The complexes can then be detected and/or quantified. A subject can then be identified as having PTI, AKI, etc. based on a comparison of the detected/quantified/measured levels of biomarkers to one or more reference controls as described herein.

In one method, a first, or capture, binding agent, such as an antibody that specifically binds the protein biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker (or to the bound capture antibody) is then used to detect binding of the polypeptide biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In a more specific embodiment, a biotinylated lectin that specifically binds a biomarker can be added to a patient sample and a streptavidin labeled fluorescent marker that binds the biotinylated lectin bound to the biomarker is then added, and the biomarker is detected.

In another embodiment, the assay is a competitive binding assay, wherein labeled protein biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

Solid phase substrates, or carriers, that can be effectively employed in such assays are well known to those of skill in the art and include, for example, 96 well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of several different protein biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, pre-determined, location on the substrate. Methods for performing assays employing such arrays include those described, for example, in US Patent Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminescence technology, can be used. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis.

In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

In several embodiments, the biomarkers of the present invention may be detected by means of an electrochemiluminescent assay developed by Meso Scale Discovery (Gaithersburg, MD). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ~620 nm, eliminating problems with color quenching. See U.S. Pat. Nos. 7,497,997; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

The proteins of the present invention can be detected by other suitable methods. Detection paradigms that can be employed to this end include optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In particular embodiments, the protein biomarker proteins of the present invention can be captured and concentrated using nano particles. In a specific embodiment, the proteins can be captured and concentrated using Nanotrap® technology (Ceres Nanosciences, Inc. (Manassas, VA)). Briefly, the Nanotrap platform reduces pre-analytical variability by enabling biomarker enrichment, removal of high-abundance analytes, and by preventing degradation to highly labile analytes in an innovative, one-step collection workflow. Multiple analytes sequestered from a single sample can be concentrated and eluted into small volumes to effectively amplify, up to 100-fold or greater depending on the starting sample volume (Shafagati, 2014; Shafagati, 2013; Longo, et al., 2009), resulting in substantial improvements to downstream analytical sensitivity.

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, CA.), Invitrogen Corp. (Carlsbad, CA), Affymetrix, Inc. (Fremong, CA), Zyomyx (Hayward, CA), R&D Systems, Inc. (Minneapolis, MN), Biacore (Uppsala, Sweden) and Procognia (Berkshire, UK). Examples of such protein biochips are described in the following patents or published patent applications: U.S. Pat. Nos. 6,537,749; 6,329,209; 6,225,047; 5,242,828; PCT International Publication No. WO 00/56934; and PCT International Publication No. WO 03/048768.

In a particular embodiment, the present invention comprises a microarray chip. More specifically, the chip comprises a small wafer that carries a collection of binding agents bound to its surface in an orderly pattern, each binding agent occupying a specific position on the chip. The set of binding agents specifically bind to each of the one or more one or more of the biomarkers described herein. In particular embodiments, a few micro-liters of, for example, blood, serum or plasma are dropped on the chip array. Protein biomarkers present in the tested specimen bind to the binding agents specifically recognized by them. Subtype and amount of bound mark is detected and quantified using, for example, a fluorescently-labeled secondary, subtype-specific antibody. In particular embodiments, an optical reader is used for bound biomarker detection and quantification. Thus, a system can comprise a chip array and an optical reader. In other embodiments, a chip is provided.

### III. DETECTION/MEASUREMENT OF NUCLEIC ACID MARKERS

Nucleic acids may be sequenced using sequencing methods such as next-generation sequencing, high-throughput sequencing, massively parallel sequencing, sequencing-by-synthesis, paired-end sequencing, single-molecule sequencing, nanopore sequencing, pyrosequencing, semiconductor sequencing, sequencing-by-ligation, sequencing-by-hybridization, RNA-Seq, Digital Gene Expression, Single Molecule Sequencing by Synthesis (SMSS), Clonal Single Molecule Array (Solexa) shotgun sequencing, Maxim-Gilbert sequencing, primer walking, and Sanger sequencing.

Sequencing methods may comprise targeted sequencing, whole-genome sequencing (WGS), lowpass sequencing, bisulfite sequencing, whole-genome bisulfite sequencing (WGBS), or a combination thereof. Sequencing methods may include preparation of suitable libraries. Sequencing methods may include amplification of nucleic acids (e.g., by targeted or universal amplification, such as PCR).

Sequencing reads can be obtained from various sources including, for example, whole genome sequencing, whole exome-sequencing, targeted sequencing, next-generation sequencing, pyrosequencing, sequencing-by-synthesis, ion semiconductor sequencing, tag-based next generation sequencing semiconductor sequencing, single-molecule sequencing, nanopore sequencing, sequencing-by-ligation, sequencing-by-hybridization, Digital Gene Expression (DGE), massively parallel sequencing, Clonal Single Molecule Array (Solexa/Illumina), sequencing using PacBio, and Sequencing by Oligonucleotide Ligation and Detection (SOLiD).

In some embodiments, sequencing comprises modification of a nucleic acid molecule or fragment thereof, for example, by ligating a barcode, a unique molecular identifier (UMI), or another tag to the nucleic acid molecule or fragment thereof. Ligating a barcode, UMI, or tag to one end of a nucleic acid molecule or fragment thereof may facilitate analysis of the nucleic acid molecule or fragment thereof following sequencing. In some embodiments, a barcode is a unique barcode (i.e., a IMI). In specific embodiments, a barcode is non-unique, and barcode sequences can be used in connection with endogenous sequence information such as the start and stop sequences of a target nucleic acid (e.g., the target nucleic acid is flanked by the barcode and the barcode sequences, in connection with the sequences at the beginning and end of the target nucleic acid, creates a uniquely tagged molecule).

Sequencing reads may be processed using methods such as de-multiplexing, de-deduplication (e.g., using unique molecular identifiers, UMIs), adapter-trimming, quality filtering, GC correction, amplification bias correction, correction of batch effects, depth normalization, removal of sex chromosomes, and removal of poor-quality genomic bins.)

In various embodiments, sequencing reads may be aligned to a reference nucleic acid sequence. In one example, the reference nucleic acid sequence is a human reference genome. As examples, the human reference genome can be hg19, hg38, GrCH38, GrCH37, NA12878, or GM12878.

### IV. METHODS OF TREATMENT

Treatment for AKI can include, but are not limited to, renal replacement therapy, angiotensin-converting-enzyme inhibitors, Ang II type I (ATI) blockers, corticosteroids, immunosuppressants, artemisinin and derivatives thereof, drag-reducing polymers, endothelin subtype A receptor antagonists (pro)renin receptor antagonists and 5-HT₃-targeting drugs.

In particular embodiments, a treatment comprises renal replacement therapy and can include, but is not limited to, hemodialysis, peritoneal dialysis, hemofiltration and renal transplantation.

A kidney treatment can also include angiotensin-converting-enzyme (ACE) inhibitor, an Ang II type I (ATI) blocker, corticosteroids, or an immunosuppressant.

In other embodiments, a treatment comprises artemisinin and derivatives thereof. Examples includes claims 1-14 of WO2014/090306. Another examples is the derivative Artesunate. See also U.S. Patent Application Publication No. 2008/0139642 and WO2010/110747.

In a specific embodiment, a treatment comprises a drag-reducing polymer (DRP). WO2013055702 describes methods for treating AKI using DRPs: DNA, RNA, PEO, polyacrylamide, hyaluronic acid, hyaluronate, rhamnogalactogalacturonan, aloe vera extract, polyethyleneimine (with hydrophilic pendant groups), glucosaminoglycans, other polyglycans, polyvinylformamide, polyphosphates, polyvinylamine, polyvinylalcohol, polyvinylpyrrolidone, polyacrylic acid, polyacrylamide, or combinations of the foregoing.

WO2014138738 describes methods of treating AKI using endothelin subtype A receptor (ETA) receptor antagonist such as atrasentan. The term “atrasentan” or “atra” OR “ABT-627” refers to (2R,3R,4S)-4-(1,3-benzodioxol-5-yl)-1-[2-(dibutylamino)-2-oxoethyl]-2-(4-methoxyphenyl)pyrrolidine-3-carboxylic acid salts thereof such as the HCl salt of atrasentan. The term “endothelin subtype A receptor antagonist” or “ETA receptor antagonist” or “ETA receptor inhibitor” refers to any compound that inhibits the effect of ET-1 signaling through the endothelin subtype A receptor. Examples of ETA receptor antagonists include, but are not limited to, ambrisentan, atrasentan, avosentan, BMS 193884, BQ-123, CI-1020, clazosentan, darusentan, edonentan, S-0139, SB-209670, sitaxsentan, TA-0201, tarasentan, TBC 3711, tezosentan. YM-598, ZD-1611, ZD-4054, and salts, esters, prodrugs, metabolites, tautomers, racemates and enantiomers thereof.

In further embodiments, a treatment comprises a (pro) renin receptor (PRR) antagonist. In WO2016/106080, PRR antagonists can be polypeptides or small molecules. Examples of functional PRR antagonist polypeptides include, but are not limited to, SEQ ID NOS:1-4 in WO2016/106080.

WO2022099027 describes the use of 5-HT₃-targeting drugs for treatment of acute kidney injury. Such drugs comprise one or more of ondansetron, granisetron, dolasetron, palonosetron, alosetron, cilansetron, tropisetron, ramosetron, or a pharmaceutically-acceptable salt or solvate of any of the preceding.

Kidney treatments include, but are not limited to, renal replacement therapy, angiotensin-converting-enzyme inhibitors, Ang II type I (ATI) blockers, corticosteroids, immunosuppressants, artemisinin and derivatives thereof, drag-reducing polymers, endothelin subtype A receptor antagonists (pro)renin receptor antagonists and 5-HT₃-targeting drugs.

In particular embodiments, a treatment comprises renal replacement therapy and can include, but is not limited to, haemodialysis, peritoneal dialysis, hemofiltration and renal transplantation.

A kidney treatment can also include angiotensin-converting-enzyme (ACE) inhibitor, an Ang II type I (ATI) blocker, corticosteroids, or an immunosuppressant.

In other embodiments, a treatment comprises artemisinin and derivatives thereof. Examples includes claims 1-14 of WO2014/090306. Another examples is the derivative Artesunate. See also U.S. Patent Application Publication No. 2008/0139642 and WO2010/110747.

In a specific embodiment, a treatment comprises a drag-reducing polymer (DRP). WO2013055702 describes methods for treating AKI using DRPs: DNA, RNA, PEO, polyacrylamide, hyaluronic acid, hyaluronate, rhamnogalactogalacturonan, aloe vera extract, polyethyleneimine (with hydrophilic pendant groups), glucosaminoglycans, other polyglycans, polyvinylformamide, polyphosphates, polyvinylamine, polyvinylalcohol, polyvinylpyrrolidone, polyacrylic acid, polyacrylamide, or combinations of the foregoing.

WO2014138738 describes methods of treating AKI using endothelin subtype A receptor (ETA) receptor antagonist such as atrasentan. The term “atrasentan” or “atra” OR “ABT-627” refers to (2R,3R,4S)-4-(1,3-benzodioxol-5-yl)-1-[2-(dibutylamino)-2-oxoethyl]-2-(4-methoxyphenyl)pyrrolidine-3-carboxylic acid salts thereof such as the HCl salt of atrasentan. The term “endothelin subtype A receptor antagonist” or “ETA receptor antagonist” or “ETA receptor inhibitor” refers to any compound that inhibits the effect of ET-1 signaling through the endothelin subtype A receptor. Examples of ETA receptor antagonists include, but are not limited to, ambrisentan, atrasentan, avosentan, BMS 193884, BQ-123, CI-1020, clazosentan, darusentan, edonentan, S-0139, SB-209670, sitaxsentan, TA-0201, tarasentan, TBC 3711, tezosentan, YM-598, ZD-1611, ZD-4054, and salts, esters, prodrugs, metabolites, tautomers, racemates and enantiomers thereof.

In further embodiments, a treatment comprises a (pro) renin receptor (PRR) antagonist. In WO2016/106080, PRR antagonists can be polypeptides or small molecules. Examples of functional PRR antagonist polypeptides include, but are not limited to, SEQ ID NOS:1-4 in WO2016/106080.

WO2022099027 describes the use of 5-HT₃-targeting drugs for treatment of acute kidney injury. Such drugs comprise one or more of ondansetron, granisetron, dolasetron, palonosetron, alosetron, cilansetron, tropisetron, ramosetron, or a pharmaceutically-acceptable salt or solvate of any of the preceding.

### VI. KITS

In another aspect, the present invention provides kits for detecting one or more biomarkers. The exact nature of the components configured in the inventive kit depends on its intended purpose. In one embodiment, the kit is configured particularly for human subjects.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

In various embodiments, the present invention provides a kit comprising: (a) one or more internal standards suitable for measurement of one or more biomarkers including by any one or more of mass spectrometry, antibody method, antibodies, lectins, nucleic acid aptamer method, nucleic acid aptamers, immunoassay, ELISA, immunoprecipitation, SISCAPA, Western blot, PCR (qPCR, digital PCR, etc.) or combinations thereof; and (b) reagents and instructions for sample processing, preparation and biomarker measurement/detection. The kit can further comprise (c) instructions for using the kit to measure biomarkers in a sample obtained from the subject.

In particular embodiments, the kit comprises reagents necessary for processing of samples and performance of an assay. In a specific embodiment, the assay is an immunoassay such as an ELISA. Thus, in certain embodiments, the kit comprises a substrate for performing the assay (e.g., a 96-well polystyrene plate). The substrate can be coated with antibodies specific for a biomarker protein(s). In a further embodiment, the kit can comprise a detection antibody including, for example, a polyclonal antibody/ies specific for a biomarker protein(s) conjugated to a detectable moiety or label (e.g., horseradish peroxidase). The kit can also comprise a standard, e.g., a human protein standard. The kit can also comprise one or more of a buffer diluent, calibrator diluent, wash buffer concentrate, color reagent, stop solution and plate sealers (e.g., adhesive strip).

In particular embodiments, the kit may comprise a solid support, such as a chip, microtiter plate (e.g., a 96-well plate), bead, or resin having protein biomarker capture reagents attached thereon. The kit may further comprise a means for detecting the protein biomarkers, such as antibodies, and a secondary antibody-signal complex such as horseradish peroxidase (HRP)-conjugated goat anti-rabbit IgG antibody and tetramethyl benzidine (TMB) as a substrate for HRP. In other embodiments, the kit can comprise magnetic beads conjugated to the antibodies (or separate containers thereof for later conjugation). The kit can further comprise detection antibodies, for example, biotinylated antibodies or lectins that can be detected using, for example, streptavidin labeled fluorescent markers such as phycoerythrin. The kit can be configured to perform the assay in a singleplex or multiplex format.

The kit may be provided as an immuno-chromatography strip comprising a membrane on which the antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of the sample.

In a specific embodiment, a kit comprises one or more of (a) magnetic beads for conjugating to antibodies that specifically bind biomarker proteins of interest; (b) monoclonal antibodies that specifically bind the biomarker proteins of interest; (c) biotinylated immunoglobulin G detection antibodies; (d) biotinylated lectins that specifically bind the biomarker proteins of interest; and (e) streptavidin labeled fluorescent marker.

In certain embodiments, a subject can be diagnosed by adding a biological sample (e.g., peripheral blood) from the patient to the kit and detecting the relevant protein biomarkers conjugated with antibodies and/or lectins, specifically, by a method which comprises the steps of (i) collecting serum from the patient; (ii) adding peripheral blood from patient to a diagnostic kit; and, (iii) detecting the protein biomarkers conjugated with antibodies/lectins. If the biomarkers are present in the sample, the antibodies/lectins will bind to the sample, or a portion thereof. In other kit and diagnostic embodiments, peripheral blood will not be collected from the patient (i.e., it is already collected). In other embodiments, the sample may comprise a urine, blood, plasma sweat, tissue, or a clinical sample.

The kit can also comprise a washing solution or instructions for making a washing solution, in which the combination of the capture reagents and the washing solution allows capture of the protein biomarkers on the solid support for subsequent detection by, e.g., antibodies/lectins or mass spectrometry. In a further embodiment, a kit can comprise instructions for suitable operational parameters in the form of a label or separate insert. For example, the instructions may inform a consumer about how to collect the sample, etc. In yet another embodiment, the kit can comprise one or more containers with protein biomarker samples, to be used as standard(s) for calibration or normalization. Detection of the markers described herein may be accomplished using a lateral flow assay.

In certain embodiments, the kit comprises reagents and components necessary for performing an electrochemiluminescent ELISA.

In some embodiments, the kit comprises a reagent that permits quantification of one or more of the nucleic acid markers described herein. In some embodiments, the kit comprises: (i) at least one reagent that allows quantification (e.g., determining the abundance, concentration or level) of an expression product of one or more of nucleic acid markers in a biological sample; and optionally (ii) instructions for using the at least one reagent. The kit can further comprise reagents for detection/measurement of other biomarkers.

A nucleic acid-based detection kit may include a primer or probe that specifically hybridizes to a target polynucleotide. The kit can further include a target biomarker polynucleotide to be used as a positive control. Also included may be enzymes suitable for amplifying nucleic acids including various polymerases (reverse transcriptase, Taq, Sequenase™, DNA ligase etc., depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe.

In a more specific embodiment, the kit is provided as a PCR kit comprising primers that specifically bind to one or more of the nucleic acid biomarkers described herein. The kit can further comprise substrates and other reagents necessary for conducting PCR (e.g., quantitative real-time PCR, digital PCR). The kit can be configured to conduct singleplex or multiplex PCR. The kit can further comprise instructions for carrying out the PCR reaction(s). In specific embodiments, the biological sample obtained from a subject may be manipulated to extract nucleic acid. In a further embodiment, the nucleic acids are contacted with primers that specifically bind the target biomarkers to form a primer:biomarker complex. The complexes can then be amplified and detected/quantified/measured to determine the levels of one or more biomarkers. The subject can then be identified as having myocardial injury based on a comparison of the measured levels of one or more biomarkers to one or more reference controls.

The reagents described herein, which may be optionally associated with detectable labels, can be presented in the format of a microfluidics card, a chip or chamber, a microarray or a kit adapted for use with the assays described in the examples or below, e.g., RT-PCR, Q PCR, digital PCR techniques described herein.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Analysis of the Human Kidney Transcriptome and Plasma Proteome Identifies Novel Biomarkers of Proximal Tubule Maladaptation to Injury

Acute kidney injury (AKI) is a common complication during hospitalization and can affect 15%-20% of hospitalized patients (1). Patients with AKI have a twofold increase in the risk of in-hospital death and a fourfold increase in the risk of developing chronic kidney disease (CKD) or experiencing CKD progression (2, 3). Emerging evidence from mouse ischemia-reperfusion injury (IRI) models of AKI suggests that kidney ischemia results in significant transcriptional changes in the proximal tubule (PT) (4). Although most PT cells under AKI stress can be fully repaired, a distinct subpopulation of PT cells enters a maladaptive, senescent phenotype that may fail to repair, leading to inflammation and fibrosis (4). This pathophysiological process suggests that PT maladaptation may mediate AKI progression, incomplete recovery, and subsequent development of CKD.

Despite the increasingly detailed knowledge derived from mouse AKI models, there may be important distinctions between humans and mice in renal tubular responses to injury (5). Histologically, frank necrosis is commonly seen in mouse kidneys after severe ischemia; however, this is uncommon in patients with AKI from acute tubular injury (6). The distinctions between mouse AKI models and hospitalized AKI patients may be one of the reasons why promising therapies derived from preclinical animal models have failed to translate into therapeutic success in human trials (7-10). Unfortunately, until the recent launch of the Kidney Precision Medicine Project (KPMP) (11), large-scale tissue interrogation studies of human AKI have been lacking. In addition, the invasive nature of kidney biopsy and the lack of noninvasive assessment of PT maladaptation have created an opportunity to identify noninvasive markers of PT maladaptation and to associate such markers with adverse clinical endpoints of AKI. Therefore, in this study, the present inventors aimed to investigate transcriptional changes in PT cells in response to injury in hospitalized patients with AKI. In addition, the present inventors developed a multiomics approach that integrates the kidney transcriptome and plasma proteome to identify biomarkers of PT maladaptation and determine their associations with severe AKI in patients undergoing cardiac surgery. The present inventors hypothesized that maladaptive PT cells that are enriched in proinflammatory and profibrotic pathways would be observed in hospitalized patients with AKI caused by diverse etiologies. The present inventors also postulated that in patients undergoing cardiac surgery, plasma proteins linked to the transcriptomic signatures of maladaptive PT cells would be associated with the development of severe AKI, as well as post-AKI kidney atrophy in mouse models of AKI.

### Materials and Methods

Study design The first goal of this study was to elucidate the transcriptomic landscape of PT cells in humans with AKI by performing snRNA-seq analysis of hospitalized participants with AKI in the KPMP study cohort. KPMP is an NIDDK-sponsored, ongoing, prospective, observational cohort study of participants with AKI and CKD receiving kidney biopsies (11). Participants with AKI were recruited if they developed AKI during hospitalization, defined as an increase in serum creatinine by 50% from their baseline, defined as the nearest outpatient serum creatinine levels 7-365 days before hospitalization, and had baseline eGFR>45 mL/min/1.73 m². Biopsies were obtained from 13 hospitalized participants with AKI who consented to research biopsies at 4 recruitment sites across the United States: Johns Hopkins Hospital, Yale-New Haven Hospital, University of Pittsburgh Medical Center, and Columbia University Medical Center. Additional biopsies were obtained from 4 hospitalized participants with COVID-19-associated AKI at Johns Hopkins Hospital. Healthy reference tissues were obtained from nontumor regions of kidney tissue after tumor nephrectomy in 3 participants and from intraoperative kidney biopsy in 4 participants undergoing urological procedures for nephrolithiasis removal in the HuBMAP consortium at Washington University at St. Louis. All samples were collected after informed consent and with the approval of the local ethics committees. Tissue processing and single-nucleus isolation were performed at Washington University at St. Louis according to the published protocol of the KPMP consortium (12)^{(preprint)}. SnRNA-seq data from 6 of 17 participants with AKI were published in a kidney atlas study by the KPMP consortium (12)^{(preprint)}, whereas data from the other 11 participants with AKI are new.

The present inventors' next goal was to link the maladaptive response in kidney tissue to the plasma proteome in order to identify potential biomarkers of PT maladaptation and determine their associations with AKI. Although it may be more biologically plausible to discover biomarkers in the urine, which the tubular cells directly face, the coverage of proteomic profiling may be low when the overall urine protein concentration is low. In addition, a recent study demonstrated that many biomarkers of kidney injury, which were originally discovered in the urine, can be measured in the blood and may have better prognostic performance (54). The present inventors used samples from the TRIBE-AKI adult study cohort, a longitudinal prospective cohort study of adults who underwent cardiac surgery in 6 academic centers in North America from July 2007 to December 2010 (53). Patients were recruited before cardiac surgery if they were at high risk of developing post-operative AKI and were prospectively followed from enrollment until death, loss to follow-up, or development of end-stage kidney disease. Of note, although the TRIBE-AKI study was among the first few pioneering studies to explore the prognostic values of kidney disease biomarkers in AKI, the proteomic data is newly generated, and this is the first investigation of proteomic data with AKI in this cohort.

The present inventors validated the proteomic findings in 2 independent cohorts. The first validation cohort was the pediatric cardiac surgery study cohort, a prospective cohort of children who underwent cardiac surgery for the repair of congenital heart disease at 3 academic centers in North America from 2007 to 2010. Children were excluded if they had a history of kidney transplantation or dialysis (55). The second validation cohort comprised marathon runners participating in the 2015 Hartford Marathon (Connecticut, US) (34). Adult runners with normal body mass indexes (18.5-24.9 kg/m²) and at least 3 years of running experience and regular training were included. Runners were excluded if they had any history of kidney disease or participated in another marathon within 4 weeks before the 2014 Hartford Marathon.

The present inventors validated the gene expression of biomarkers of maladaptive PT and PT cells at healthy states in 3 KPMP participants with AKI who were recently enrolled and were not included in the biomarker discovery phase, as well as in kidney autopsy tissues from a cohort of critically-ill patients with AKI published by Hinze et al (33). The present inventors further validated the gene expression of biomarkers of maladaptive PT and PT cells at healthy states in 2 mouse models with different repair capacities after IRI (repair and atrophy models), as the present inventors previously reported (35), and in a mouse model of AAN presenting as AKI-to-CKD transition. All human subject research studies were approved by each institution's research ethics board, and all animal protocols were approved by the Yale University Animal Care and Use Committee.

Human snRNA-seg data library preparation, preprocessing, and analysis. The present inventors used the Cell Ranger 7.0 pipeline to align snRNA-seq FASTQ files to the human hg38 reference genome. The present inventors then used CellBender to remove ambient RNA contamination and DoubletDetection to remove doublets (56, 57). The present inventors used Seurat v4 for data preprocessing and analyses, including log normalization, scaling, clustering, dimension reduction, and examination of differential gene expression (58). After removing ambient RNA contamination and doublets, the present inventors excluded low-quality nuclei with less than 200 or more than 7,500 genes detected (12)^{(preprint)}. The present inventors removed unique molecular identifiers mapped to mitochondrial RNA from analysis and combined all samples for further processing.

To correct for batch effects, the present inventors performed data integration using reciprocal principal component analysis on 2,000 highly variable genes across each sample after log-normalization and scaling. The present inventors then performed principal component analysis in the integrated dataset, and chose the 15 principal components determined by using the ElbowPlot function in Seurat. The present inventors further performed dimension reduction to a uniform manifold approximation and projection (UMAP) plot and performed Louvain clustering using a resolution of 0.5 after k-nearest neighbor embedding. Using an approach similar to that of a recent snRNA-seq study of human AKI autopsy kidney tissues (33), each cluster of a major kidney cell type was further integrated and clustered. Subclusters that expressed canonical markers of more than 2 distinct cell types, which may represent doublets, were removed. The present inventors repeated this integration-clustering step iteratively until no subcluster of doublets could be identified. The canonical markers of major kidney cell types used are: PT: CUBN and SLC5A12; thin limb: SLC44A5; thick ascending limb: UMOD and SLC12A1; distal convoluted tubule: SLC12A3; connecting tubule: CALB1; principal cell: AQP2, SCNN1G; intercalated A cell, SLC4A1; intercalated B cell, SLC26A4; podocyte, NPHS2; endothelium, FLT1; fibroblast, ACTA2 and COL1A1; and immune cell, CD163, IL7R, NKG7, MS4A1, MZB1, HLA-DQA1, and MS4A2.

The present inventors focused the present inventors' further analysis on PT subclusters. The present inventors obtained lists of differentially-expressed genes for each PT subcluster by comparing gene expression in that subcluster to that of other subclusters using the Wilcoxon test (FindAllMarkers function in Seurat) and accounting for multiple comparisons using the false discovery rate. The present inventors included all differentially-expressed genes and performed GSEA using the FGSEA package (59)^{(preprint)}. The present inventors defined PT maladaptation as PT cells with near-complete dedifferentiation and enrichment of proinflammatory and profibrotic genes and pathways.

Gene regulatory network analysis. To further investigate the gene regulation landscape in PT cells at healthy and diseased states and during AKI, the present inventors used pySCENIC for gene regulatory network analysis (60). The present inventors focused on the analysis of PT nuclei data in participants with AKI and retained 4,247 highly variable genes (minimal dispersion 0.4 using Scanpy) for analysis (61). The present inventors generated coexpression networks via Epoch and cell regulatory networks of PT nuclei with the human hg38 reference genome for cis-regulatory analysis (32). The present inventors obtained the gene-motif ranking of 10 kb around the transcription start site (62). The present inventors further plotted the top 10% of transcription factor-target gene pairs using the igraph package and performed community detection using the Louvain clustering algorithm. All snRNA-seq analyses were performed in R 4.1.2 and Python 3.7.

Sample collection and proteomic measurements in study cohorts. Sample collection and processing in the 3 cohorts has been previously described in detail (34, 53, 55). For the TRIBE-AKI adult and pediatric cardiac surgery cohorts, blood samples were collected preoperatively and postoperatively after cardiac surgery. The postoperative samples were collected within 6 hours after surgery. For the TRIBE-AKI adult cohort, a subset of 54 participants had urine collected pre- and postoperatively for urine proteomic profiling. For the marathon cohort, blood samples were collected 24 hours before (prerace) and within 30 minutes after the marathon (postrace). Blood and urine samples were centrifuged and stored at −80 °C until measurement. Proteomic measurements were performed using the Slow Off-Rate Modified Aptamer (SOMA)-based capture array on preoperative and the first postoperative plasma or urine samples. Plasma and urine samples were shipped to SomaLogic (Boulder, CO) for identification and quantification of low-abundance plasma proteins by SOMAscan, which uses easily quantifiable, chemically-modified oligonucleotides as binding reagents for proteins and protein complexes (63). Protein analyte measurements underwent the SOMAscan data standardization and normalization process and were matched to their corresponding genes.

Outcome definition for the primary analysis in TRIBE-AKI adult participants. The primary outcome of the present inventors' analysis is severe (KDIGO stage 2 and 3) AKI. The present inventors define stage 2 AKI as a twofold increase in serum creatinine from baseline to the peak postoperative value. The present inventors define stage 3 AKI as a threefold increase in serum creatinine from baseline to the peak postoperative value or requiring kidney replacement therapy.

Validation of biomarker gene expression in kidney tissues from individuals with AKI. The prospective KPMP cohort allowed us to internally validate the present inventors' biomarker findings in 3 recently enrolled participants whose snRNA-seq data were not included in the biomarker discovery phase. In these 3 participants, snRNA-seq libraries were prepared using the approach described earlier. The present inventors used the metadata from 17 participants with AKI from the discovery phase as reference, and predicted PT cell subtypes using pySingleCellNet, a random forest-based classifier (64). In addition, the present inventors downloaded an snRNA-seq library of post-mortem kidney tissues from an independent cohort of 8 critically-ill patients with AKI published by Hinze et al (GSE210622) (33), and performed clustering of PT cells using the approach discussed by these investigators. Biomarker gene expression patterns were explored in the internal and external validation cohorts.

Animal surgery and experimental protocol. To further validate the findings from the present inventors' proteomic investigation, the present inventors compared 2 mouse models of IRI (repair and atrophy) that the present inventors previously reported (35) and examined the gene expression of 4 biomarkers associated with (mal)adaptive kidney repair following IRI. Additionally, the present inventors determined the gene expression kinetics in a mouse model of AAN that presents as AKI-to-CKD transition. All animal protocols were approved by the Yale University Animal Care and Use Committee. Male C57BL/6 (Envigo, Indianapolis, IN) wild-type mice (aged 9-11 weeks) were used in this work.

To establish the unilateral IRI (atrophy) model, warm renal ischemia was induced using a nontraumatic microaneurysm clip (FST Micro Clamps, Foster City, CA) on the left renal pedicle for 27 minutes, leaving the right kidney intact. To establish the unilateral IRI with contralateral nephrectomy (repair) model, the right kidney was surgically removed at the time of left kidney ischemia, as the present inventors have previously described (35). Mice were sacrificed on days 1, 7, 14, and 30 after surgery (n=9-10 per timepoint for each model). Control mice were sacrificed and represented as day 0 (n=9).

To establish a mouse model of AAN, 14 mice were treated with a single 5 mg/kg body weight dose of aristolochic acid (Sigma-Aldrich) intraperitonially, and 4 mice were treated with vehicle. Blood was collected on days 3, 7, 10, 14, and 21 after injection. Seven AAN-treated mice were sacrificed and kidneys were harvested on days 7 and 21 post-injection. Mice treated with the vehicle were used as experimental controls and were sacrificed on day 21 post-injection. Blood urea nitrogen (BUN) was measured using Stanbio Diagnostic Set (Fisher Scientific). In addition, the present inventors downloaded a publicly available snRNA-seq dataset of mouse AAN kidneys published by Lu et al (37), and compared biomarker gene expression between the kidney fibrosis phase (day 28) and baseline.

Quantitative PCR analysis. Whole-kidney RNA was extracted with an RNeasy Mini kit (Qiagen, Germantown, MD) and reverse transcribed using the iScript cDNA Synthesis Kit (Bio-Rad Laboratories). Gene expression analysis was determined by quantitative real-time PCR using an iCycler iQ (Bio-Rad Laboratories) and normalized to hypoxanthine-guanine phosphoribosyltransferase (Hprt). Primers used include mouse Col23a1 (forward: GGCATAAGTGATCCTCAGACATAA (SEQ ID NO:1) and reverse: AGTTGGCGCATCCCATAAA (SEQ ID NO:2)), Enpp6 (forward: GGAACACATGACCGTGTATGA (SEQ ID NO:3) and reverse: TCTCTCGACTCTCTGCTATGAA (SEQ ID NO:4)), Tgfb2 (forward: AGAGGGATCTTGGATGGAAATG (SEQ ID NO:5) and reverse: TGAGGACTTTGGTGTGTTGAG (SEQ ID NO:6)), Proc (forward: CCTCAAACGAGACACAGACAGACTTAG (SEQ ID NO:7) and reverse: GATCATACTCACCAAGCCTCAC (SEQ ID NO:8)), and Hprt (forward: CAGTACAGCCCCAAAATGGT (SEQ ID NO:9) and reverse: CAAGGGCATATCCAACAACA (SEQ ID NO:10)). Data were expressed using the comparative threshold cycle (ΔCT) method and mRNA ratios were given by 2^{-ΔCT}.

Statistical analysis. The present inventors report descriptive characteristics of 322 TRIBE-AKI participants with and without the primary outcome using medians (IQR) and proportions. The present inventors compare descriptive characteristics using Wilcoxon tests and chi-square tests.

To identify biomarkers of PT maladaptation, the present inventors first selected differentially-expressed genes that have an average log 2-fold change of more than 0.25 in the maladaptive PT cell subcluster and selected corresponding proteins measured using aptamer assays by SOMAscan. In mouse AK PT maladaptation after IRI is unable to fully repair. Therefore, the present inventors hypothesize that cardiac surgery will result in PT injury and subsequent maladaptation and that a postoperative increase in protein biomarkers from maladaptive PT cells will be associated with development of severe AKI in the hospital. The present inventors retained proteins if their median levels were higher postoperatively and the median postoperative levels were higher in patients with severe AKI than in patients without severe AKI by Wilcoxon tests. The present inventors then manually examined gene expression across all cells and selected those genes specific to the maladaptive PT subcluster. This process yielded 4 biomarkers of PT maladaptation for further analysis. The present inventors additionally determined correlations between preoperative maladaptation biomarkers and baseline eGFR using Spearman correlation.

To identify biomarkers of PT cells at healthy states that decreased in maladaptive states, the present inventors applied a similar workflow. These biomarkers can be viewed as “inverse” biomarkers of PT maladaptation (i.e., lower levels indicate more severe PT maladaptation). The present inventors first identified upregulated genes with log 2-fold changes>0.25 in healthy PT cells compared to diseased PT subclusters and extracted the corresponding proteins. The present inventors retained proteins if their median levels were lower postoperatively and if the median postoperative protein levels were lower in patients with severe AKI than in patients without severe AKI by Wilcoxon tests. Similarly, the present inventors manually examined gene expression across all cells and selected those that were specifically enriched in healthy PT cells and downregulated in maladaptive PT cells. This process yielded 5 biomarkers of PT cells at healthy states for further analysis.

To determine associations between biomarkers and the primary outcome in TRIBE-AKI adult participants, the present inventors used univariable and multivariable logistic regression models with sequential adjustment of covariates. Model 1 was the univariable model including postoperative biomarkers only. Model 2 adjusted for biomarkers, age, sex, and race. Model 3 adjusted for the variables in model 2 as well as baseline eGFR, hypertension, diabetes mellitus, myocardial infarction, and heart failure. Model 4 adjusted for the variables in model 3 as well as the baseline urine albumin to creatinine ratio. Model 5 was derived from model 4 with additional adjustment for preoperative biomarker values.

The present inventors next determined if the addition of biomarkers of PT maladaptation and PT cells at healthy states can further improve the prediction of severe AKI beyond other kidney disease biomarkers (kidney injury molecule-1, neutrophil gelatinase-associated lipocalin, and soluble urokinase plasminogen activator receptor) and other clinical variables (age, sex, Black race, pre-operative eGFR, albuminuria, hypertension, diabetes, myocardial infarction, and heart failure). The present inventors compared the area under the curve of the logistic regression models using 1,000 bootstrap samples.

For the subset of 54 TRIBE-AKI adult participants who had urine collected for proteomic profiling, the present inventors compared the protein levels in the first postoperative urine samples with the preoperative samples. For validation in pediatric cardiac surgery participants, the present inventors compared protein levels in the first postoperative samples versus preoperative samples. For validation in marathon runners, the present inventors compared protein levels in the immediate postrace samples versus the prerace samples. The present inventors performed these comparisons using pairwise Wilcoxon tests.

For the present inventors' mouse models of IRI followed by repair and atrophy, the present inventors used two-way ANOVA (GraphPad Prism 8) for model comparison to test whether there was a difference between the models and in the time course, followed by Sidak post-tests for subgroup comparison at each timepoint. In addition, the present inventors determined the Pearson correlation of biomarker genes with fibrosis marker genes in the recovery phase of AKI (days 7, 14, and 30). For the mouse model of AAN, the present inventors used ANOVA followed by Tukey tests for comparisons across subgroups, and Student t tests to determine the difference between gene expression in the AKI (7 day) and CKD (21 day) phases compared with baseline. The present inventors conducted a complete case analysis and considered a two-sided P value<0.05 as statistically significant. All statistical analyses were performed using R version 4.1.2.

Data and materials availability. The snRNA-seq human AKI data have been deposited in the KPMP data repository (https://atlas.kpmp.orp/repository/) and the code used for analysis is available at GitHub (https://github.com/ywen1407/snRNAseq_AKI_aptamer_PT_maladaptation). The proteomic data analyzed in this work is not publicly available because widespread sharing of TRIBE-AKI study data was not stipulated in the ethics approval for the study. The TRIBE-AKI principal investigator (Chirag R. Parikh, chirag.parikh@jhmi.edu) may be contacted for data requests.

### Results

Single-nucleus RNA sequencing reveals diverse PT cell phenotypes in human AKI. The present inventors used single-nucleus RNA sequencing (snRNA-seq) to profile 120,985 nuclei from 17 participants with AKI and 7 healthy participants, including data from 13 participants (6 with AKI and 7 healthy references) that were published previously and 11 participants with AKI whose data were unpublished (Supplemental Data Si, not shown) (12)^{(preprint)}. The median number of unique molecular identifiers per nucleus was 2,941 (IQR: 2,069-3,620) and the median number of genes detected per nucleus was 1,720 (IQR: 891-2843) (Supplemental Data S2, not shown). Among participants with AKI, 11 (64.7%) were male and 6 (35.3%) self-identified as Black. The baseline serum creatinine was 1.23 mg/dL, 13 (76.5%) participants had stage 3 AKI, and 16 (94.1%) participants received a kidney biopsy after their serum creatinine level had peaked, with a median time from AKI diagnosis to biopsy at 7 (IQR: 4-10) days. Fourteen (82.4%) participants had acute tubular injury from a variety of causes, such as nonsteroidal anti-inflammatory drugs, antibiotics, sepsis, COVID-19, rhabdomyolysis, and oxalate nephropathy. The demographic and clinical characteristics of participants with AKI and healthy references are presented in detail in Supplemental Data Si, not shown.

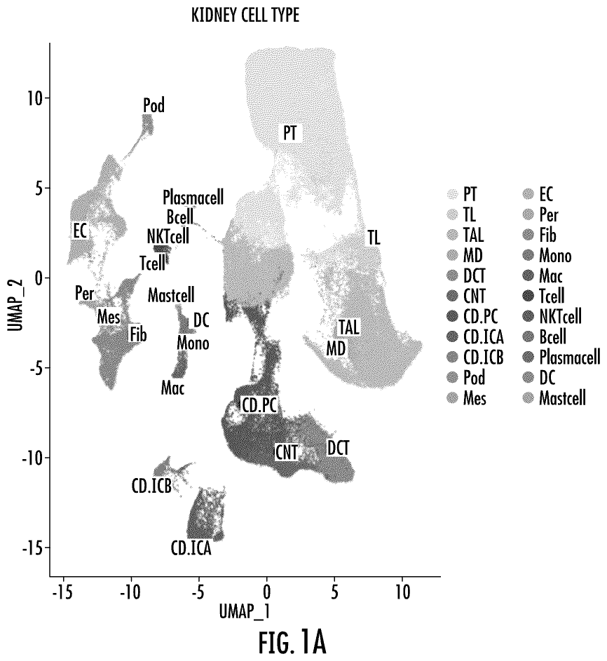

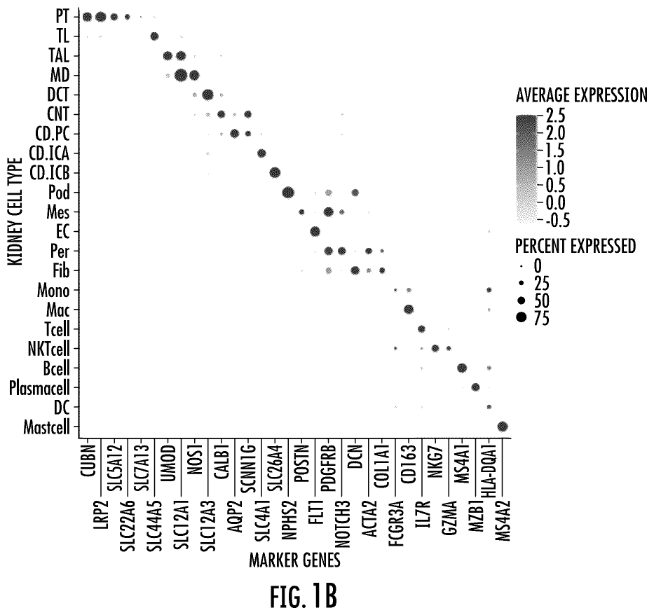

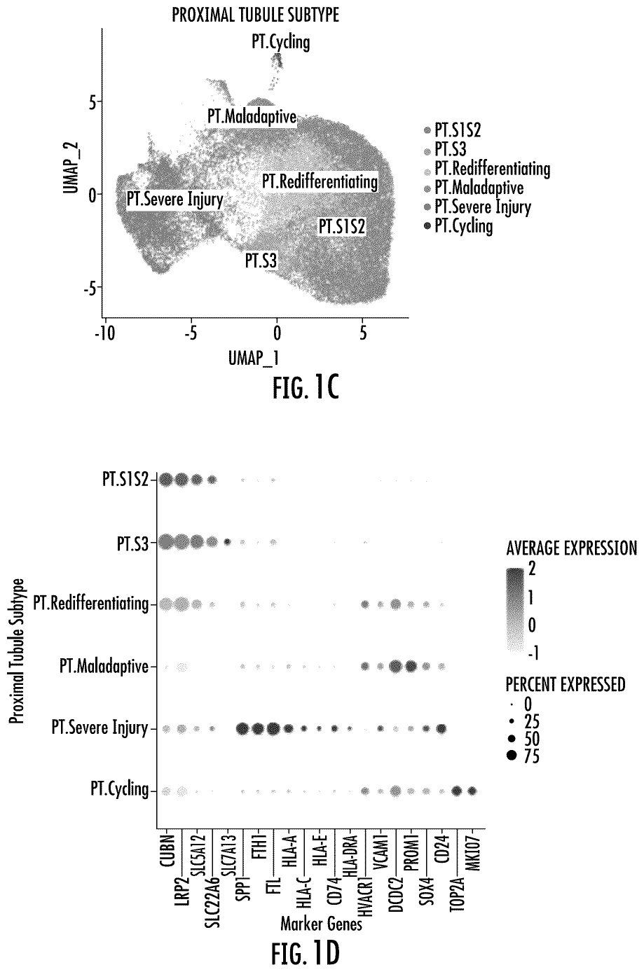

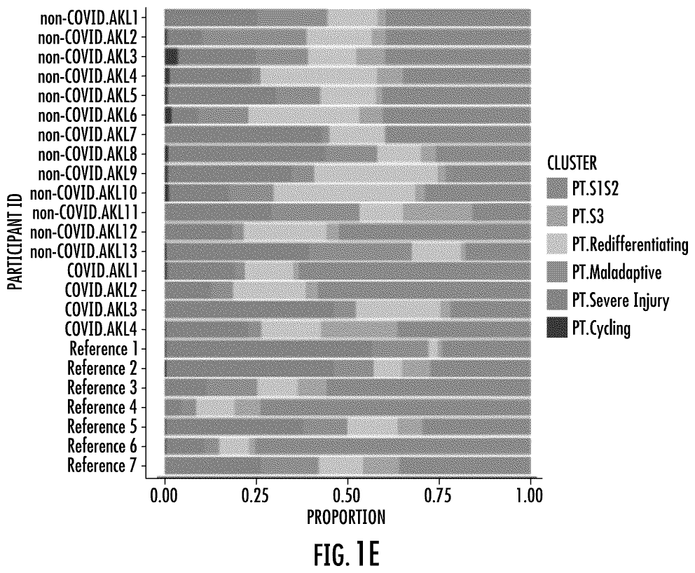

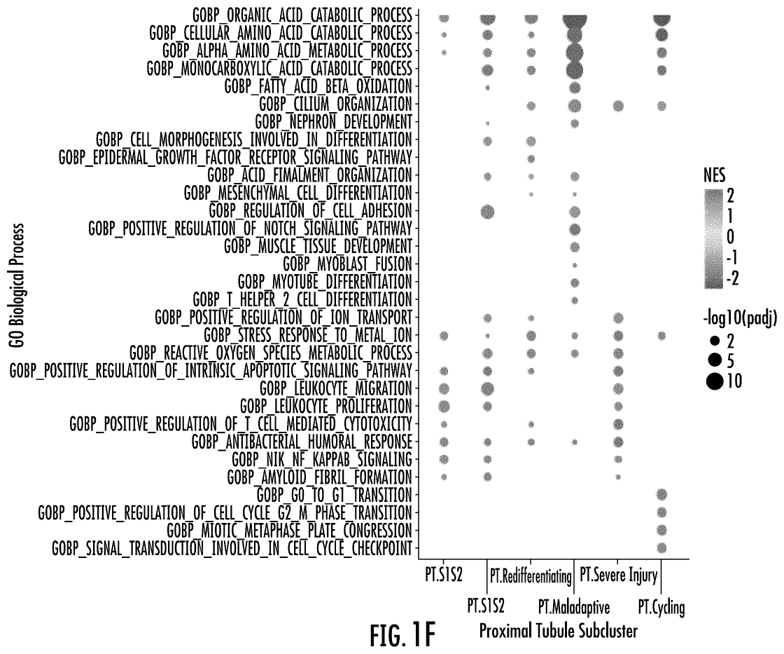

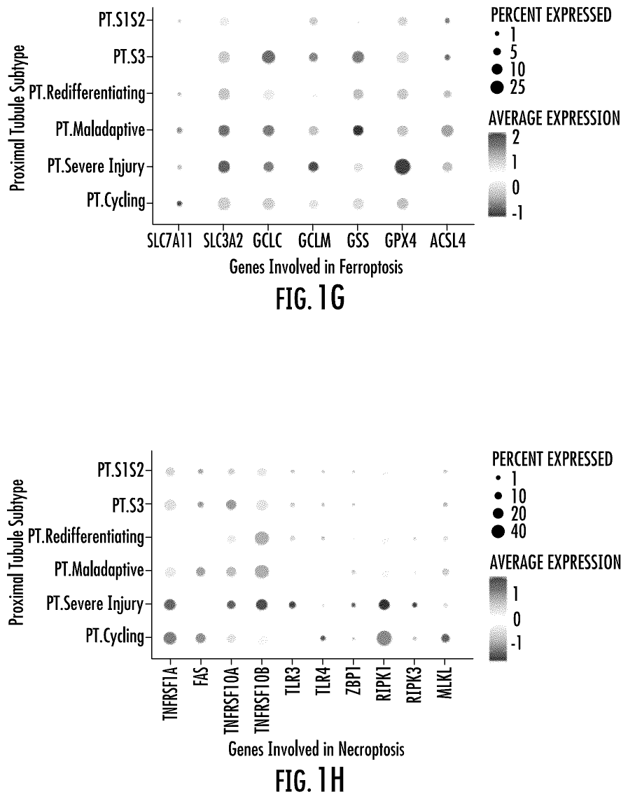

Using unsupervised clustering, the present inventors identified clusters of all major kidney, stromal, and immune cell types in participants with and without AKI (FIG. 1A, 1B). The present inventors focused the present inventors' analysis to PT cells, which included 6 subclusters, 2 of which (PT.S1S2 and PT.S3) were enriched in mature PT markers (SLC5A12, SLC22A6, SLC22A7, and SLC7A13) (FIG. 1C-1E, Supplemental Data S3, not shown). Gene set enrichment analysis (GSEA) of these 2 subclusters demonstrated an enrichment in gene ontology terms involved in the physiological function of PT cells, such as organic anion transport and fatty acid metabolism (FIG. TF, Supplemental Data S4, not shown), consistent with their relatively healthy states. The proportion of cells belonging to the healthy PT subclusters was greater in samples from healthy references but diminished in samples from AKI participants, indicating a significant shift in the phenotypes of PT cells during AKI (FIG. 1E).

Among the 4 subclusters of PT cells with decreased expression of mature PT markers (FIG. 1D, Supplemental Data S3, not shown), the present inventors observed a subcluster of PT cells enriched in markers of proliferation (TOP2A and MKI-67). The other 3 subclusters exhibited 2 distinct phenotypes based on marker gene expression and pathway analysis. In severely injured PT cells, there was significant upregulation of markers of cellular stress (SPP1), iron hemostasis (FTH1 and FTL), injury (SOX4 and CD24), MHC class I (HLA-A, HLA-C and HLA-E), and MHC class II (CD74 and HLA-DRA). Consistent with these changes in marker gene expression, GSEA of this subcluster indicated prominent immune system activation and apoptosis associated with severe injury. The other 2 PT subclusters had lost their differentiated states and expressed the injury markers HAVCR1 and VCAM1. The expression of canonical PT markers (CUBN and LRP2) and solute transporters was markedly diminished in one cluster (PT.maladaptive), indicating its advanced dedifferentiated state. GSEA of these 2 subclusters of dedifferentiated PT cells demonstrated enrichment in nephron regeneration as well as in Notch signaling pathways (FIG. 1F, Supplemental Data S4, not shown). The terminally dedifferentiated subcluster was additionally enriched in genes associated with immune activation and migration, extracellular matrix adhesion, and fibroblast activation. The present inventors refer to the terminally dedifferentiated subcluster hereafter as maladaptive PT because it exhibits similar marker gene expression (e.g., VCAM1, HAVCR1, and DCDC2) and pathway enrichment as the mouse “failed to repair” PT described in a previous study in mice (4). In addition to the proinflammatory and profibrotic signature, the present inventors observed upregulation of ACSL4 and downregulation of GCLC, GSS, and GPX4 (FIG. 1G), indicating potential activation of the ferroptosis pathway and loss of the capacity to remove toxic polyunsaturated fatty acid-phospholipid hydroperoxides (13). There was also a concurrent increase in the expression of necroptosis markers, such as death receptors (FAS, TNFRSF10A, and TNFRSF10B) and the necroptotic executioner MLKL (FIG. 1H).

Despite various etiologies and severities of AKI and heterogeneity in the timing of kidney biopsy, the present inventors observed similar enrichment of severely injured and maladaptive PT cells as well as diminished proportions of healthy PT cells in participants with AKI, indicating potentially common responses to insult at the transcriptional level in PT cells (FIG. 1E). Interestingly, one participant (non-COVID AKI #8) had completely recovered renal function at the time of kidney biopsy; however, at the tissue level, there was still a considerable proportion of injured and maladaptive PT cells. Furthermore, injured and maladaptive PT cells were seen in healthy controls. Preclinical studies showed that failed-to-repair, maladaptive PT cells constitute approximately 5% of PT cells in mice. The proportion becomes higher with increased age and comorbidities, such as diabetes (4, 14). The high prevalence of severely injured and maladaptive PT in the present inventors' healthy controls could be due to advanced aging, subclinical vascular disease, and hemodynamic fluctuation during anesthesia (Supplemental Data Si, not shown).

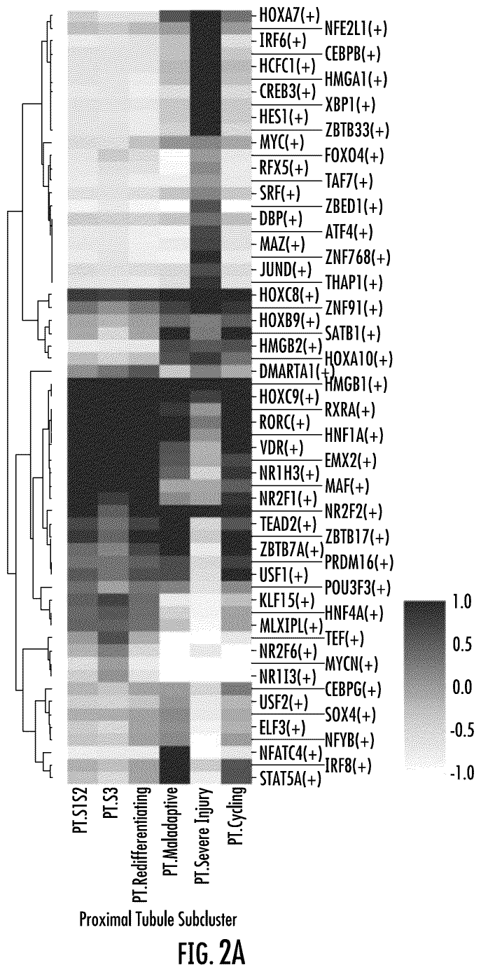

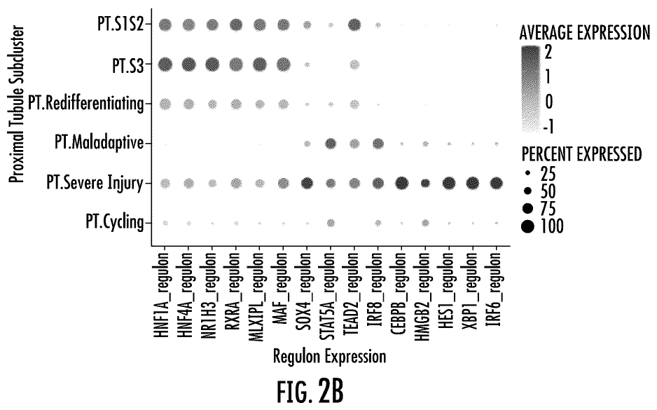

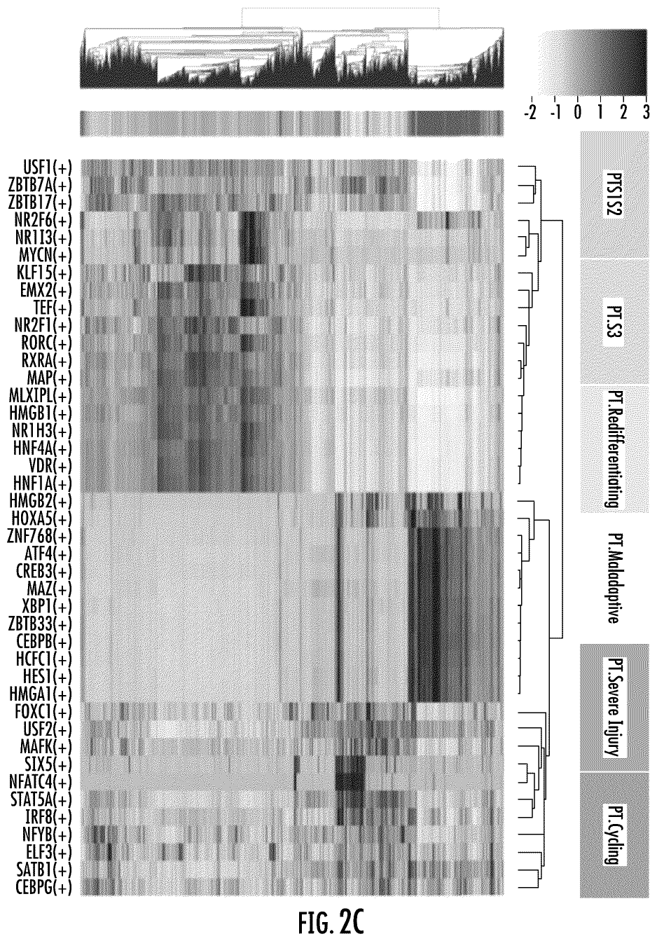

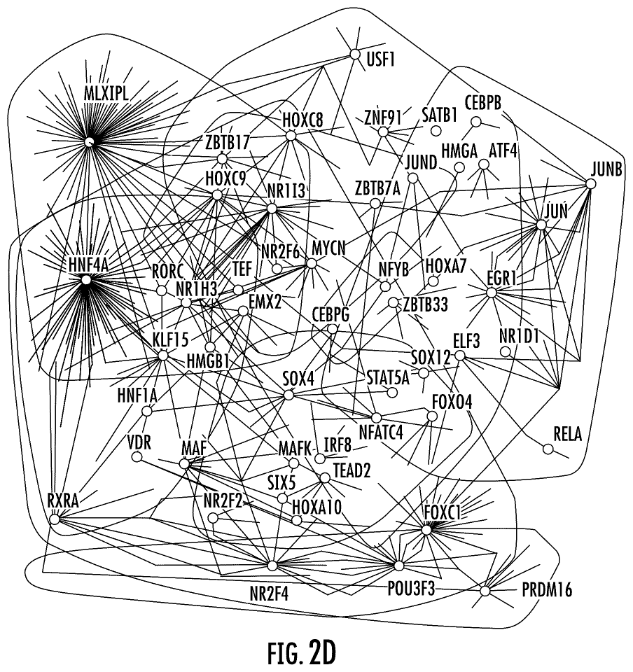

Gene regulatory network analysis identifies distinct regulators activated in PT cells in healthy and diseased states. The present inventors next used pySCENIC to explore whether the gene regulatory structure (i.e., regulons) in PT cells in diseased states was deranged compared to the healthy state in participants with AKI. Similar to several mouse AKI studies, the present inventors observed enrichment of regulons (HNF1A, HNF4A, NR1H3, MAF, RXRA, and MLXIPL) involved in promoting and maintaining PT cell differentiation, stabilizing mitochondrial structure, and maintaining mitochondrial lipid metabolism in PT cells at healthy states (FIG. 2A, Supplemental Data S5-S6, not shown) (15-20). RXRA is also known to protect tubules from oxidative stress and prevent the AKI-to-CKD transition (21). Regulon enrichment diminished significantly as PT cells progressed toward the maladaptive state. Specifically, maladaptive PT cells were enriched in SOX4, a key regulator promoting nephrogenesis, and in STAT5A, a regulator that drives abnormal tubular cell growth (22-24). Consistent with the profibrotic and proinflammatory signatures from GSEA, maladaptive PT upregulated TEAD2, a regulator governing the epithelial-to-mesenchymal transition, and IRF8, a transcription factor promoting inflammatory responses (25-27). As a comparison, severely injured PT cells activated regulators of the response to an inflammatory milieu (CEBPB and HMGB2) as well as regulators driving endoplasmic reticulum stress (HES1 and XBP1) and apoptosis in diseased states (HMGB2 and IRF6) (28 31). The expression of regulons across PT cells in healthy and diseased states was consistent with the cellular enrichment estimated from pySCENIC (FIG. 2B). Examining the 10 most-enriched regulons across each PT subcluster demonstrated clear distinctions in the regulatory networks across PT cells at different states of health (FIG. 2C). In summary, these results consistently demonstrated that severely injured PT cells activate an intrinsic program driving cellular stress and apoptosis. In contrast, maladaptive PT cells lost physiological function and simultaneously reactivated regeneration programs and mediators of inflammation and fibrosis.

The present inventors next examined the top 10% of transcription factor-target gene pairs reconstructed by Epoch and applied Louvain unsupervised clustering for community detection of key regulatory modules (32). Each community represented a group of transcription factors coregulating a set of target genes (FIG. 2D). Consistent with previous findings, the present inventors observed coregulation of target genes by HNF4A, HNF1A, and NR1H3, all of which were enriched in healthy PT cells. In maladaptive PT cells, SOX4 and STAT5A formed a coregulation network with TEAD2 and IRF8 and governed the expression of a group of target genes, suggesting that inflammation and fibrosis are associated with activation of aberrant nephron regeneration during the repair process.

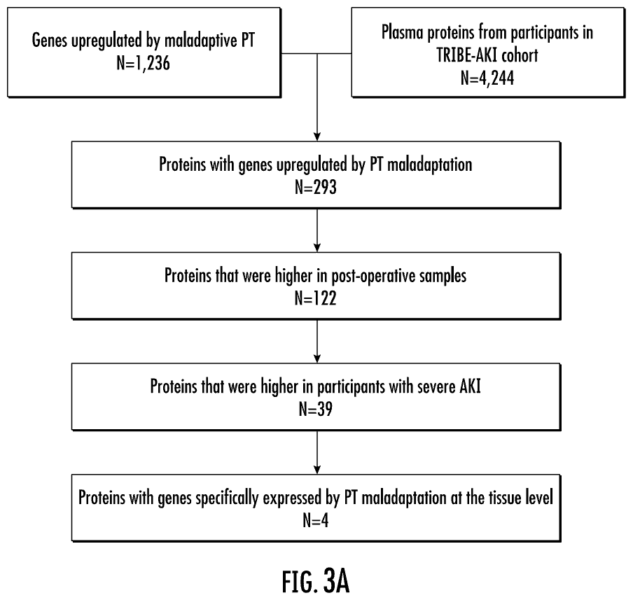

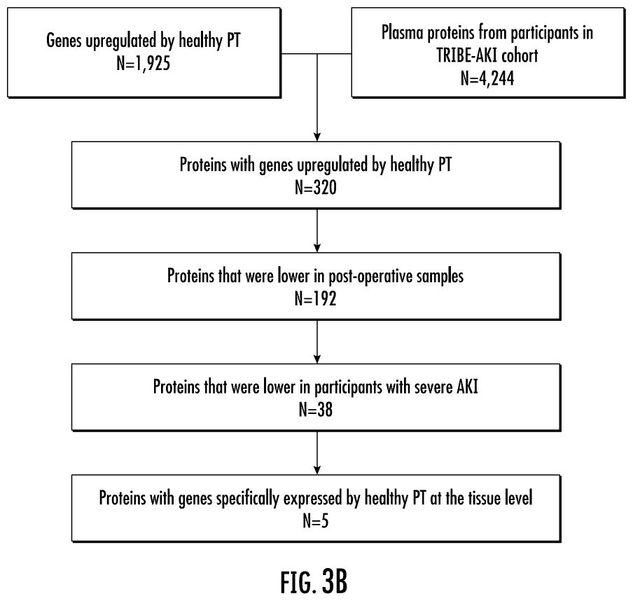

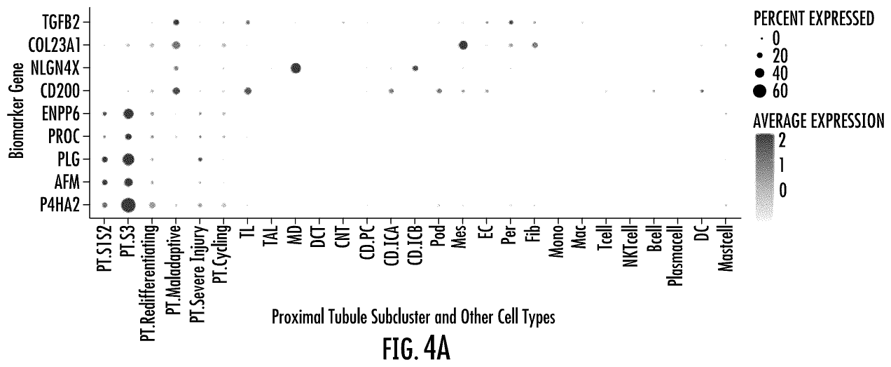

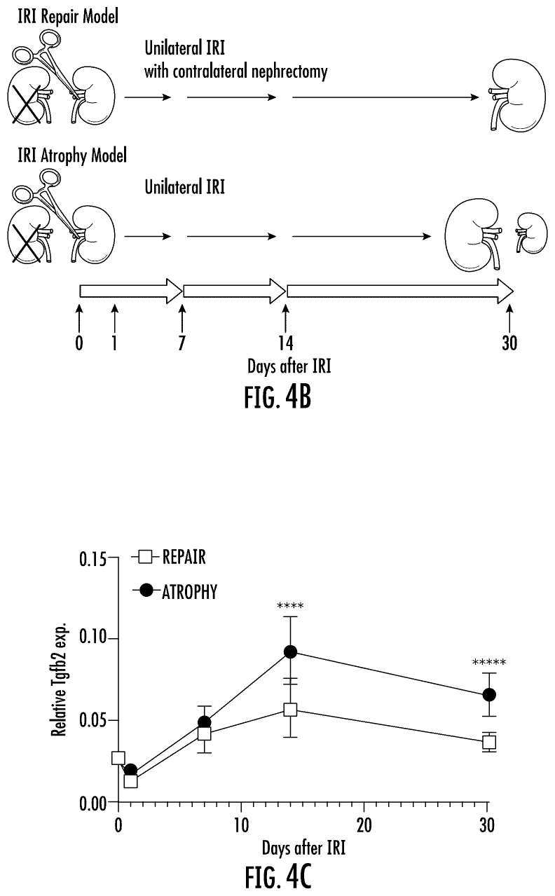

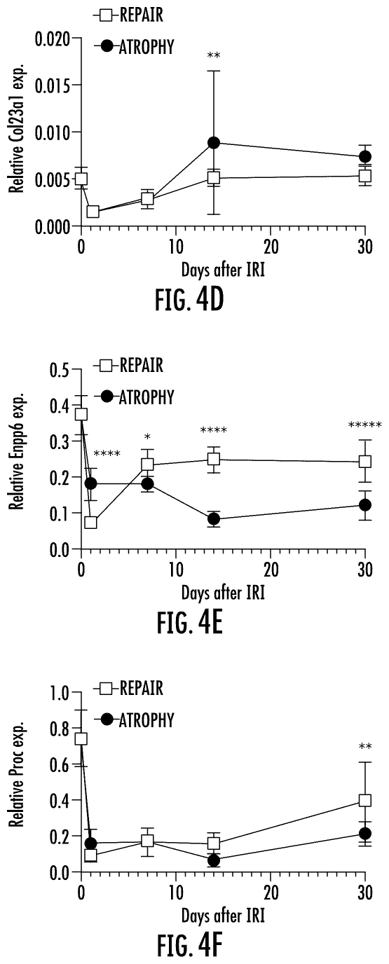

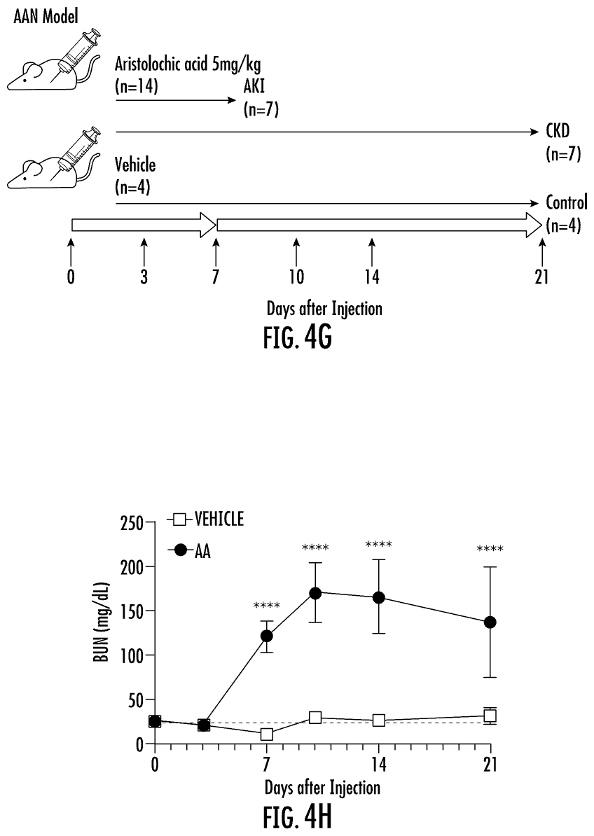

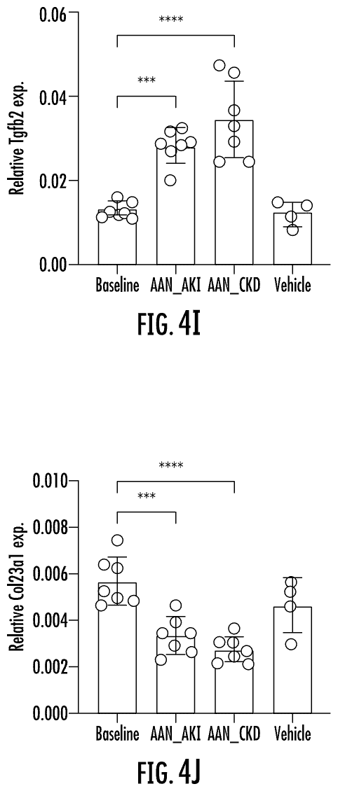

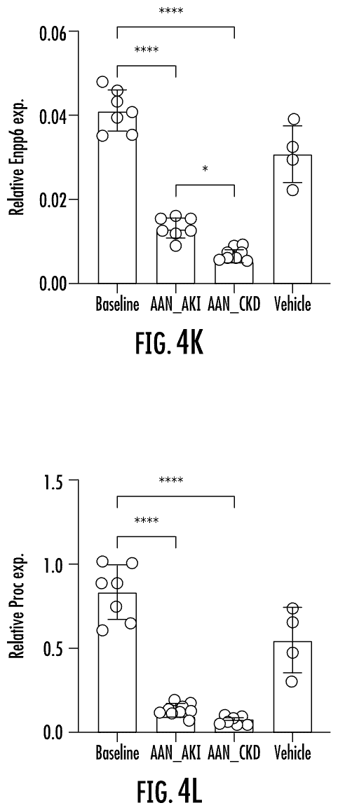

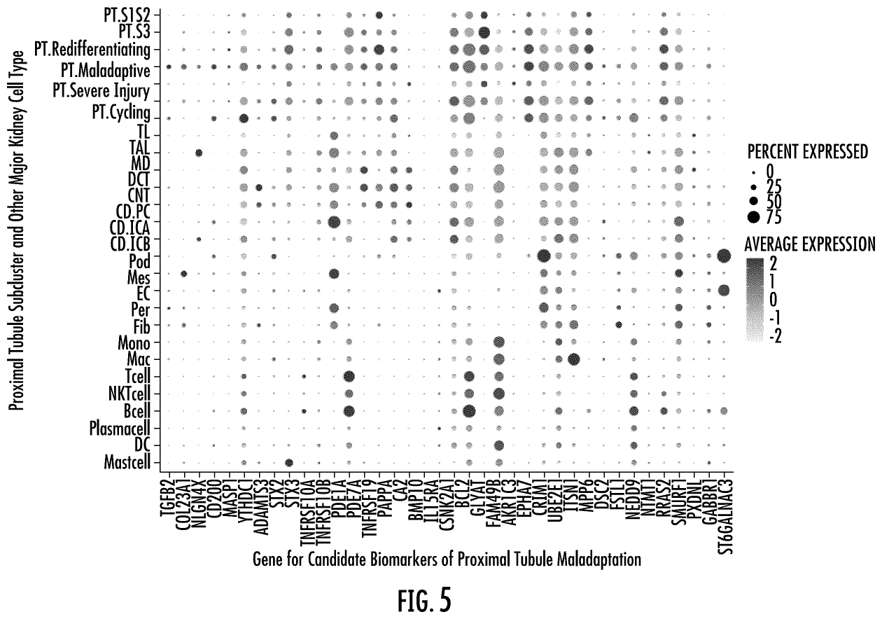

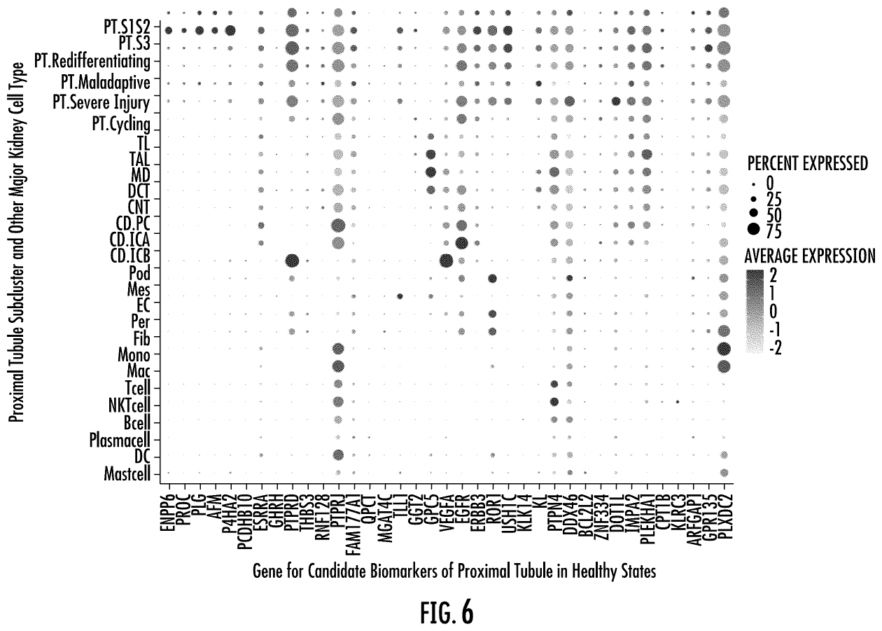

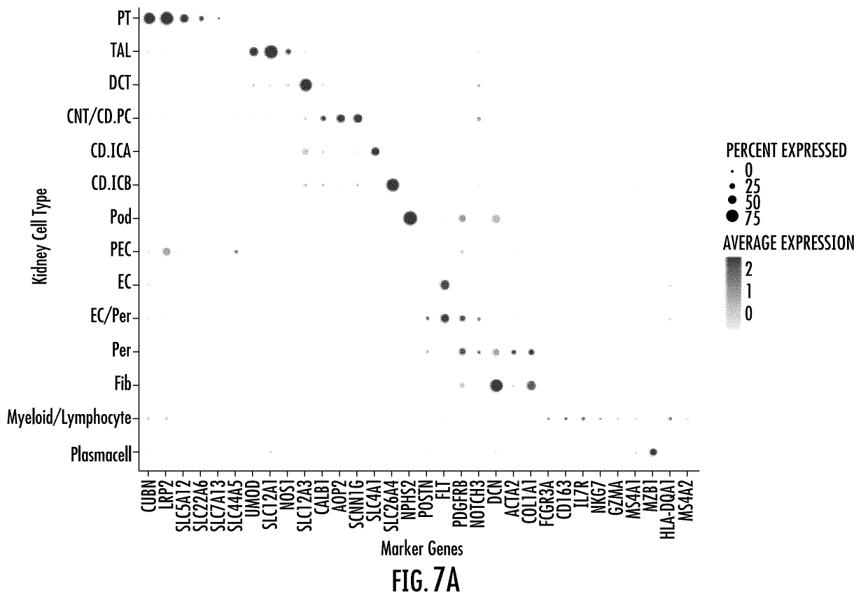

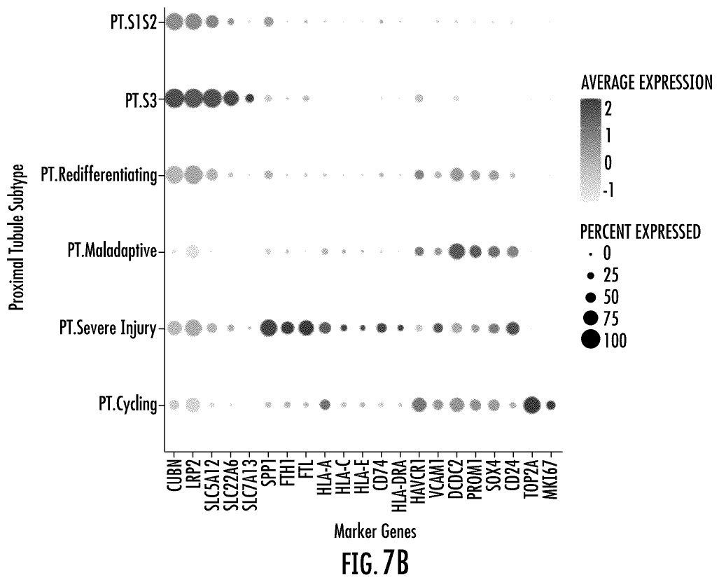

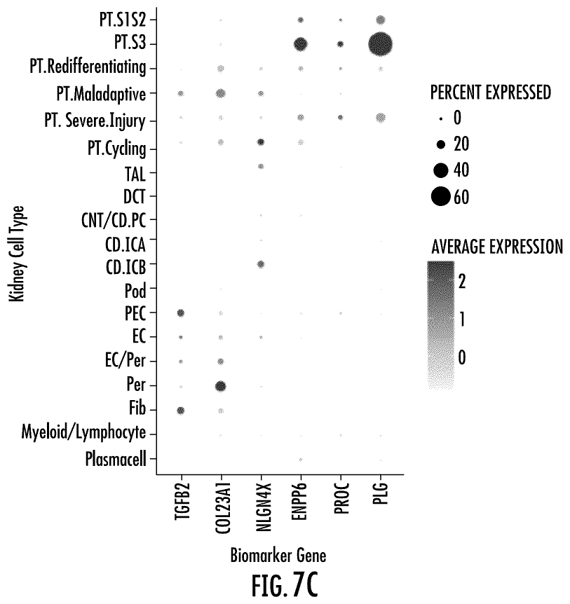

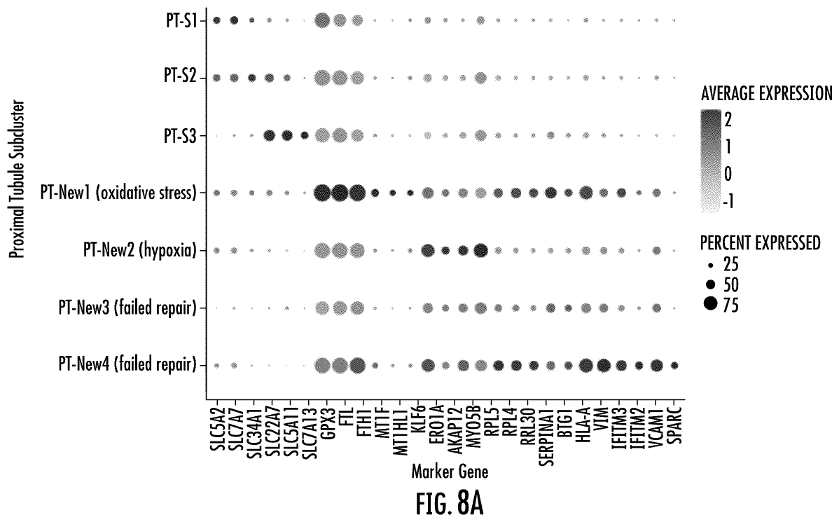

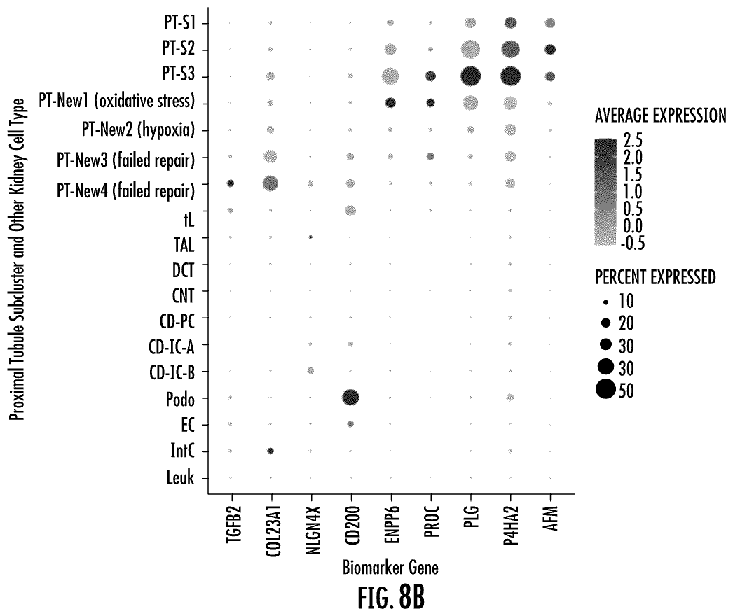

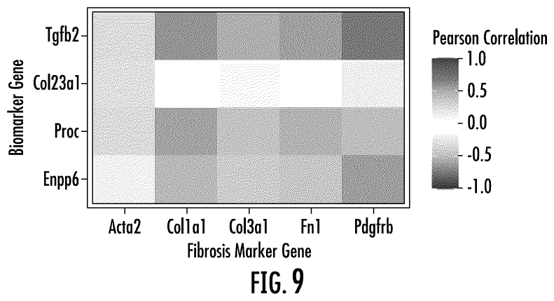

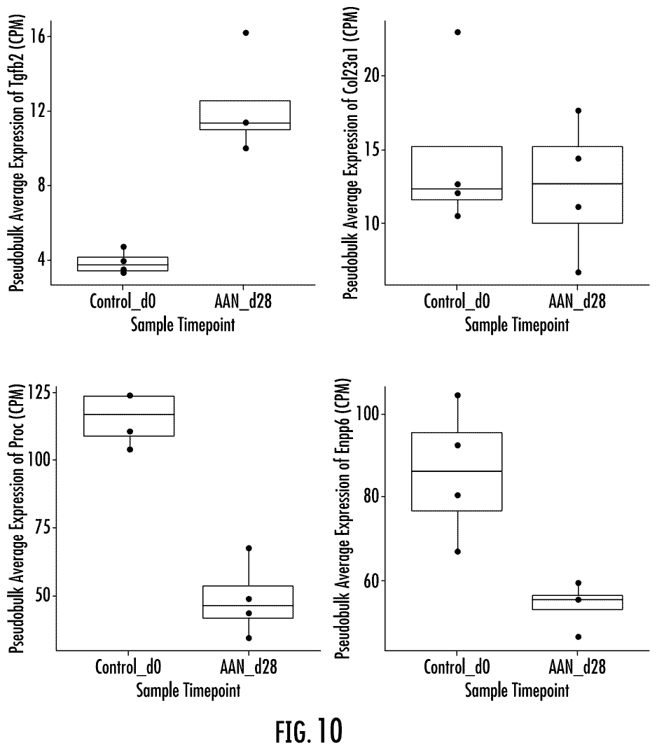

A multiomic investigation identifies biomarkers of PT maladaptation. To determine the associations between PT maladaptation and severe AKI, the present inventors next aimed to determine whether the present inventors could identify noninvasive biomarkers, such as plasma proteins, that are specific of maladaptive PT cells. Using differentially-expressed genes from maladaptive PT cells in KPMP participants with AKI and proteins measured in 322 adults undergoing cardiac surgery in the Translational Investigation of Biomarker Endpoint of AKI (TRIBE-AKI) cohort, the present inventors developed a workflow to identify candidate biomarkers of PT maladaptation (FIGS. 3A and 3B). For genes upregulated by PT maladaptation, the present inventors identified 293 genes that had plasma proteins measured using aptamer assays with SOMAscan. Among these 293 proteins, 122 displayed higher plasma levels postoperatively compared with the preoperative baseline, and 39 had significantly higher levels in patients who developed stage 2-3 AKI (hereafter referred as severe AKI). The present inventors further examined the gene expression of these 39 proteins in KPMP participants and identified 4 proteins that were relatively specific to maladaptive PT cells as candidate biomarkers of PT maladaptation (FIG. 4A, FIG. 5). The present inventors also identified 320 genes that were downregulated as PT cells progressed from healthy to maladaptive states and measured the encoded proteins using SOMAscan. Among these, 192 proteins were lower postoperatively and 38 were significantly lower in patients who developed severe AKI. The present inventors further examined expression of the genes encoding these 38 proteins in KPMP participants and identified 5 that were specifically enriched in healthy PT cells and downregulated in maladaptive PT cells (FIG. 4A, FIG. 6). The expression patterns of these proteins were consistently seen in kidney biopsy tissues in 3 recently enrolled KPMP participants with AKI who were not included in the tissue interrogation and biomarker discovery phase, as well as in postmortem kidney tissues in an independent cohort of 8 critically-ill patients with AKI (FIG. 7-8) (33).

Table 1 displays the baseline characteristics of TRIBE-AKI adult participants with and without severe AKI. Among 322 participants, 47 developed severe AK. The median age of all participants was 73 years, 71.7% were male, and 94.1% were White. The median baseline estimated glomerular filtration rate (eGFR) was 68.2 mL/min/1.73 m², and the median baseline urine albumin-creatinine ratio (ACR) was 15 mg/g. Pre- and postoperative protein levels and their fold changes are shown in Table 4 and Table 5, respectively.

Using multivariable logistic regression with sequential adjustment of covariates, the present inventors observed strong and positive associations between postoperative maladaptation biomarkers and the development of severe AKI (Table 3). NLGN4X, COL23A1, and TGFB2 were significantly associated with increased odds of severe AKI in all models, including model 5, which adjusted for preoperative biomarker levels. In general, the increase of plasma biomarkers in kidney disease may be caused by the decrease in GFR thus may lead to spurious associations (i.e., reverse causation). However, the preoperative levels of these proteins are not correlated with preoperative eGFR, suggesting a lack of reverse causation (Table 5). In a subset of 54 TRIBE participants who had urine samples collected before and after surgery for proteomic profiling, there were significant increases in urine NLGN4X and COL23A1 postoperatively but no change in TGFB2 excretion (Table 6).

Regarding the 5 biomarkers of PT cells at healthy states, the present inventors observed that postoperative ENPP6, PROC, and PLG were inversely associated with the development of severe AKI (i.e., a lower plasma level indicated a higher risk of severe AKI). P4HA2 and AFM were associated with severe AKI in univariable analysis but lost statistical significance when adjusting for comorbidities (Table 3). Three plasma biomarkers of PT cells at healthy states can also be measured in the urine by SOMAscan aptamer assays. Excretion of both PLG and AFM significantly increased after cardiac surgery, and excretion of PROC became significantly higher after being indexed to urine creatinine to account for urine dilution (Table 6), which may be due to shedding from healthy PT cells after injury.

When added to known biomarkers of kidney diseases, such as kidney injury molecule-1, neutrophil gelatinase-associated lipocalin, and soluble urokinase plasminogen activator receptor, biomarkers of PT maladaptation and PT cells at healthy states can further enhance the predictive performance for AKI (Table 7).

The present inventors validated these plasma proteins as indicators of PT maladaptation and PT cells at healthy states in 2 independent cohorts. The pediatric cardiac surgery cohort comprised 68 participants undergoing surgery for the repair of congenital heart disease, with a median age of 41 (IQR: 6-84) months, including 37 (54.4%) girls. The present inventors observed significant increases in plasma TGFB2, COL23A1, and NLGN4X (proteins of PT maladaptation), and significant decreases in plasma ENPP6, PLG, and PROC (proteins of PT cells at healthy states) after cardiac surgery, similar to the findings in their adult counterparts (Table 2, Table 4). Next, the present inventors compared these proteins in 39 participants before and immediately after running a marathon. The present inventors previously demonstrated that after completion of a marathon, runners could develop significant tubular injury, with excretion of kidney injury and inflammation biomarkers increasing by 3- to 12-fold immediately after the race (34). The median age of the marathon runners was 42 (IQR: 33-51) years, 21 (53.8%) were women, 2 (5.1%) participants had hypertension, and 1 (2.6%) had diabetes. The present inventors observed significant increases in plasma TGFB2 and NLGN4X and significant decreases in plasma PLG and PROC after the marathon, consistent with the present inventors' findings from the 2 cardiac surgery cohorts (Table 2, Table 4).

Reverse translational investigation validates biomarkers of PT maladaptation in mouse models of ischemic and toxic AKI. To further validate the associations between these proteins and the maladaptive response to AKI of different causes, the present inventors conducted a reverse translational investigation and compared the mRNA expression of the biomarker genes (Col23a1, Tgfb2, Enpp6, and Proc) in 2 different mouse models of IRI followed by either repair or atrophy, as well as in a mouse model of toxic aristolochic acid nephropathy (AAN) presenting as AKI-to-CKD transition (35). Of note, Plg and Nlgn4 were excluded from this analysis because expression of these genes is very low to undetectable at the whole-kidney level, as well as based on publicly available single-cell and snRNA-seq datasets from injured mouse kidney (4, 36).

In the setting of mouse kidney IRI, quantitative RT-PCR of whole-kidney mRNA revealed that Tgfb2 was expressed at significantly higher levels in the setting of progressive kidney fibrosis and atrophy, whereas the expression of Enpp6 and Proc (markers expressed by PT cells at healthy states) were increased in the setting of kidney repair (FIG. 4B-F). The continuous increase in gene expression of Tgfb2 and the sustained decreases in Enpp6 and Proc during progressive kidney fibrosis indicated a persistent, unresolved, maladaptive response in the kidney, beyond the immediate repair phase of IRI. Moreover, Tgfb2 had strong, positive correlations with known fibrosis markers (Col1a1, Col3a1, Fn1, Pdgfb, and Acta2), and Enpp6 and Proc had moderate to strong negative correlations with fibrosis (FIG. 9, Table 8). In the AAN model, the present inventors also observed a progressive increase in Tgfb2 expression, as well as continuing decreases in Enpp6 and Proc expression as mice progressed from AKI to CKD (FIG. 4 G-L). These quantitative PCR results are consistent with results from a different model of repeated AKI leading to CKD from aristolochic acid by Lu et al (37) and results from the present inventors' IRI models (FIG. 10). These results in mouse models of AKI are largely similar to findings from human AKI kidney biopsies and further support the association of these biomarkers with maladaptive repair after diverse causes of injury to the kidney. Of note, Col23a1 expression in both the IRI and AAN models was low. It did not differ significantly between the repair and atrophy models at 30 days after IRI, and decreased from baseline in the AAN model.

In summary, the present inventors' transcriptomic investigation demonstrated that maladaptive repair in PT cells is a shared response to injury in hospitalized patients with AKI of diverse causes. Furthermore, the present inventors identified plasma NLGN4X, COL23A1, and TGFB2 as novel biomarkers that increased in the setting of PT maladaptation after cardiac surgery, whereas plasma ENPP6, PLG, and PROC serve as novel biomarkers of PT cells at healthy states that diminish in the setting of maladaptive repair after injury.

### DISCUSSION

In this study, the present inventors used snRNA-seq analysis of kidney tissue from hospitalized participants with AKI and identified PT cells in a distinctive maladaptive state, characterized by the loss of differentiated states and physiological function and the activation of aberrant kidney regeneration signatures associated with a proinflammatory and profibrotic milieu. This maladaptive repair at the transcriptional level represents a shared response to injury in participants with AKI of diverse etiologies. By integrating the transcriptome associated with PT maladaptation with the plasma proteome from a different cohort of patients undergoing cardiac surgery, the present inventors found that increases in postoperative plasma NLGN4X, COL23A1, and TGFB2 and decreases in postoperative plasma ENPP6, PROC, and PLG were strongly associated with the development of severe AKI. Reverse translational investigations in mouse models of ischemic and toxic AKI further suggested associations between these biomarkers and maladaptive repair and fibrosis, and supported a role for these biomarkers in noninvasive assessment of PT maladaptation.

During the process of AKI progression, incomplete recovery, and AKI-to-CKD transition in mice, some injured PT cells from AKI undergo dedifferentiation but then fail to redifferentiate and recover normal function. These PT cells exhibit a maladaptive profile, lose their physiological function, enter a senescent cell-cycle arrest phase, activate programmed cell death pathways, and eventually form atrophic tubules (36, 38). In addition, maladaptive PT cells persistently produce and secrete profibrotic factors, such as TGF-β, and recruit and activate the transition of pericytes and fibroblasts into myofibroblasts, leading to production of matrix material and fibrosis (39). The present inventors' study demonstrated that PT maladaptation, characterized by enrichment in proinflammatory, profibrotic, ferroptotic and necroptotic pathways, is similarly present in human AKI. In these maladaptive PT cells, the present inventors further identified that the activation of regulators involved in maladaptive tubular cell growth, such as SOX4 and STAT5A, was accompanied by a close coregulation network with proinflammatory and profibrotic mediators (40). Whether therapeutic intervention halting this maladaptive repair process can attenuate the risk of severe AKI and the AKI-to-CKD transition requires further investigation.

PT maladaptation was widely present in participants with AKI of diverse etiologies, suggesting a shared response to injury at the tubular cell level. This is consistent with observations from mouse models of ischemic and toxic AKI, in which injured PT cells enter the senescent and maladaptive phenotype and mediate interstitial fibrosis (37, 41). In addition, a large proportion of maladaptive PT cells were observed in the participant who had completely recovered renal function at the time of kidney biopsy. In mice, serum creatinine may return to near baseline within 7 days after kidney injury; however, the maladaptive process may persist for weeks (41, 42). Taking these results together, therapies may need to be initiated and maintained for a long period of time, perhaps for months after AKI, to prevent the AKI-to-CKD transition.

Kidney tissue interrogation allows the identification of PT maladaptation for in-depth mechanistic investigation. However, the invasive nature of the kidney biopsy procedure makes it challenging for this interrogation to be applied to large cohorts of patients. This highlights the importance of developing sensitive and noninvasive biomarkers to measure this maladaptive repair process at the tissue level. These biomarkers may characterize the prevalence of PT maladaptation and establish its etiological associations with clinical complications of AKI in large cohorts of patients. They may also serve as potential pharmacodynamic endpoints for early investigation of targeted therapeutics in preventing complications of AKI.

With this motivation, the present inventors developed a workflow to integrate findings from snRNA-seq with the plasma proteome in patients at high risk of AKI from cardiac surgery. The discovery of biomarkers by integrating these 2 different cohorts, instead of using only the kidney biopsy cohort, is largely due to the small sample size of patients with available transcriptomic data. The present inventors' snRNA-seq analysis demonstrates that the PT maladaptation phenotype may be shared across different patient populations with diverse causes of AKI, further suggesting the feasibility of this integrative approach. The present inventors identified multiple biomarkers of PT maladaptation associated with AKI progression. Tubular epithelial expression of TGF-β plays a critical role in the development of interstitial fibrosis after AKI (43, 44). Although the TGF-β2 isoform is not well described in AKI, it is implicated in the epithelial-to-mesenchymal transition in cancer cells, potentially by interacting with urokinase plasminogen activator (45). X-linked neuroligin-4 (encoded by NLGN4X) and collagen XXIII α-1 (COL23A1), although not yet described in kidney disease, may be involved in cellular junction and extracellular matrix formation. These proteins may potentially arise from post-injury fibrosis (46, 47). The associations between AKI and these biomarkers of cell adhesion, migration, and extracellular matrix material are consistent with the profibrotic profile of PT maladaptation. In addition, the present inventors identified multiple novel biomarkers of the successful repair of injured PT cells. Plasminogen and protein C have been shown to ameliorate fibrosis and inflammation after renal IRI (48, 49). ENPP6 is involved in the extracellular degradation of glycerophosphocholine to provide choline intracellularly but has not yet been reported in kidney disease (50). These results suggest that the biomarkers discovered from the present inventors' study may not only have prognostic value, but may also provide potential mechanistic insights on the maladaptive repair of PT cells in the progression of AKI.

Although the present inventors could not validate the correlation between these plasma proteins and gene expression in the present inventors' kidney biopsy cohort due to its limited sample size, post-cardiac surgery changes in these biomarkers were highly consistent between the 2 cohorts of adult and pediatric patients undergoing cardiac surgery. The discrepancies in 2 biomarkers, COL23A1 and ENPP6, between marathon runners and patients undergoing cardiac surgery may be due to differences in the kidney's response to these 2 distinct insults. Although marathons may be associated with volume depletion, IRI, and heat stress, cardiac surgery may additionally involve an enhanced inflammatory milieu from cardiopulmonary bypass (51, 52). In addition, upon validating markers of PT maladaptation and PT cells at healthy states in mouse IRI models with different repair capacities as well as a model of toxic kidney injury, the present inventors found that 3 of the 4 markers were associated with maladaptive changes, interstitial fibrosis, and kidney atrophy. The consistency of the gene expression patterns in ischemic and toxic AKI models further support the stereotypical maladaptive response observed in human AKI of diverse etiologies. The low expression level of Col23a1 and its lack of correlation with fibrosis, as well as the lack of Nlgn4 and Plg expression in mouse kidneys could be due to transcriptomic differences between humans and mice and highlight the importance and benefit of direct interrogation of human kidney tissues.

The present inventors' study has many important implications. The present inventors' snRNA-seq analysis demonstrated that maladaptive repair of PT cells may be a stereotypical response to injury in participants with diverse etiologies and severities of AKI. In addition, there may be a link between maladaptive tissue regeneration and inflammation that should be explored further in mechanistic studies for potential therapeutic agent development. By integrating the kidney tissue transcriptome and plasma proteome, the present inventors discovered multiple proteins that reflect the maladaptive and healthy states of PT cells, which can be measured noninvasively to establish the etiological association between PT maladaptation and adverse outcomes of AKI in large cohorts of patients. Future studies can determine if these plasma proteins can serve as pharmacodynamic endpoints in early-stage clinical trials investigating drugs targeting PT maladaptation. The present inventors' multiomics biomarker development pipeline may also be adopted in research aiming to establish associations between diseased cell states and clinical outcomes in other kidney diseases.

The present inventors recognize several limitations of this study that are worth noting. The present inventors could not assess gene expression changes along the trajectory of PT maladaptation. This is due to the cross-sectional nature of kidney biopsy procedures following AKI diagnosis and interindividual variations of PT cells with diverse disease pathophysiology and clinical courses, which makes the use of trajectory analysis tools challenging. Proteomic and snRNA-seq analysis were performed in 2 different cohorts, making us unable to directly correlate plasma proteins with tissue gene expression. Proteomic profiling in the biomarker discovery cohort was performed using only the first postoperative plasma samples. Proteins that were released to the plasma later in the course of injury and maladaptation and proteins that were more likely to be excreted to the urine, such as kidney injury molecule-1, may not be adequately captured and may lead to false-negative results (53). Future studies may investigate whether additional biomarkers can be identified using plasma samples obtained later in the course of AKI and urine samples. Although the present inventors validated the plasma proteomic findings in 2 independent cohorts of patients at risk of kidney injury, the small sample size of the validation cohorts and the relatively lower risk for clinical AKI, compared with adult cardiac surgery patients, limit the present inventors' ability to determine associations between these proteins and clinical AKI. Plasma proteins of PT maladaptation can also originate from tissues other than the kidney. However, the correlation between kidney expression of these markers and fibrosis in mouse models of ischemic and toxic AKI were reassuring. Lastly, protein levels quantified by SOMAscan may not correlate perfectly with those measured using targeted immunoassays, necessitating further validation for implementation in research and clinical practice.

### REFERENCES

1. C. Ronco, R. Bellomo, J. A. Kellum, Acute kidney injury. The Lancet 394, 1949-1964 (2019).
2. E. A. J. Hoste, S. M. Bagshaw, R. Bellomo, C. M. Cely, R. Colman, D. N. Cruz, K. Edipidis, L. G. Fori, C. D. Gomersall, D. Govil, P. M. Honore, O. Joannes-Boyau, M. Joannidis, A.-M. Korhonen, A. Lavrentieva, R. L. Mehta, P. Palevsky, E. Roessler, C. Ronco, S. Uchino, J. A. Vazquez, E. Vidal Andrade, S. Webb, J. A. Kellum, Epidemiology of acute kidney injury in critically ill patients: the multinational AKI-EPI study. Intensive Care Med. 41, 1411-1423 (2015).
3. S. G. Coca, S. Singanamala, C. R. Parikh, Chronic kidney disease after acute kidney injury: a systematic review and meta-analysis. Kidney Int. 81, 442-448 (2012).
4. Y. Kirita, H. Wu, K. Uchimura, P. C. Wilson, B. D. Humphreys, Cell profiling of mouse acute kidney injury reveals conserved cellular responses to injury. Proc. Natl. Acad. Sci. U.S.A 117, 15874-15883 (2020).
5. S. Rosen, N. H. Samuel, Difficulties in understanding human acute tubular necrosis: Limited data and flawed animal models. Kidney Int. 60, 1220-1224 (2001).
6. Y. Wen, C. Yang, S. P. Menez, A. Z. Rosenberg, C. R. Parikh, A Systematic Review of Clinical Characteristics and Histologic Descriptions of Acute Tubular Injury. Kidney Int. Rep. 5, 1993-2001 (2020).
7. C. N. Sharp, L. J. Siskind, Developing better mouse models to study cisplatin-induced kidney injury. Am. J. Physiol. Renal Physiol. 313, F835-F841 (2017).
8. R. L. Allgren, T. C. Marbury, S. N. Rahman. L. S. Weisberg, A. Z. Fenves, R. A. Lafayette, R. M. Sweet, F. C. Genter, B. R. Kurnik, J. D. Conger, M. H. Sayegh, Anaritide in acute tubular necrosis. Auriculin Anaritide Acute Renal Failure Study Group. N. Engl. J. Med. 336, 828-834 (1997).
9. P. Pickkers, R. L. Mehta, P. T. Murray, M. Joannidis, B. A. Molitoris, J. A. Kellum, M. Bachler, E. A. J. Hoste, O. Hoiting, K. Krell, M. Ostermann, W. Rozendaal, M. Valkonen, D. Brealey, A. Beishuizen, F. Meziani, R. Murugan, H. de Geus, D. Payen, E. van den Berg, J. Arend, S.-A. Investigators, Effect of Human Recombinant Alkaline Phosphatase on 7-Day Creatinine Clearance in Patients With Sepsis-Associated Acute Kidney Injury: A Randomized Clinical Trial. JAMA 320, 1998-2009 (2018).
10. J. A. Tumlin, K. W. Finkel, P. T. Murray, J. Samuels, G. Cotsonis, A. D. Shaw, Fenoldopam Mesylate in Early Acute Tubular Necrosis: A Randomized, Double-Blind, Placebo-Controlled Clinical Trial. Am. J. Kidney Dis. 46, 26-34 (2005).
11. Kidney Precision Medicine Project. Natl Inst Diabetes Dig Kidney Bethesda MD (2018).
12. B. B. Lake, R. Menon, S. Winfree, Q. Hu, R. M. Ferreira, K. Kalhor, D. Barwinska, E. A. Otto, M. Ferkowicz, D. Diep, N. Plongthongkum, A. Knoten, S. Urata, A. S. Naik, S. Eddy, B. Zhang, Y. Wu, D. Salamon, J. C. Williams, X. Wang, K. S. Balderrama, P. Hoover, E. Murray, A. Vijayan, F. Chen, S. S. Waikar, S. Rosas, F. P. Wilson, P. M. Palevsky, K. Kiryluk, J. R. Sedor, R. D. Toto, C. Parikh, E. H. Kim, E. Z. Macosko, P. V. Kharchenko, J. P. Gaut, J. B. Hodgin, M. T. Eadon, P. C. Dagher, T. M. El-Achkar, K. Zhang, M. Kretzler, S. Jain, for the KPMP Consortium, An atlas of healthy and injured cell states and niches in the human kidneybioRxiv, 2021.07.28.454201 (2021).
13. B. R. Stockwell, Ferroptosis turns 10: Emerging mechanisms, physiological functions, and therapeutic applications. Cell 185, 2401-2421 (2022).
14. Y. Muto, P. C. Wilson, N. Ledru, H. Wu, H. Dimke, S. S. Waikar, B. D. Humphreys, Single cell transcriptional and chromatin accessibility profiling redefine cellular heterogeneity in the adult human kidney. Nat. Commun. 12, 2190 (2021).
15. S. S. Marable, E. Chung, J.-S. Park, Hnf4a Is Required for the Development of Cdh6-Expressing Progenitors into Proximal Tubules in the Mouse Kidney. J. Am. Soc. Nephrol. JASN 31, 2543-2558 (2020).
16. Y. Wang, A. H. Moser, J. K. Shigenaga, C. Grunfeld, K. R. Feingold, Downregulation of liver X receptor-alpha in mouse kidney and HK-2 proximal tubular cells by LPS and cytokines. J. Lipid Res. 46, 2377-2387 (2005).
17. G. Martovetsky, J. B. Tee, S. K. Nigam, Hepatocyte nuclear factors 4α and 1α regulate kidney developmental expression of drug-metabolizing enzymes and drug transporters. Mol. Pharmacol. 84, 808-823 (2013).
18. S. Shirota, T. Yoshida, M. Sakai, J. I. Kim, H. Sugiura, T. Oishi, K. Nitta, K. Tsuchiya, Correlation between the expression level of c-maf and glutathione peroxidase-3 in c-maf −/− mice kidney and c-maf overexpressed renal tubular cells. Biochem. Biophys. Res. Commun. 348, 501-506 (2006).
19. S. Suzuki, A. Yokoyama, E. Noro, S. Aoki, K. Shimizu, H. Shimada, A. Sugawara, Expression and pathophysiological significance of carbohydrate response element binding protein (ChREBP) in the renal tubules of diabetic kidney. Endocr. J. 67, 335-345 (2020).
20. A. K. Stoeckman, L. Ma, H. C. Towle, Mlx is the functional heteromeric partner of the carbohydrate response element-binding protein in glucose regulation of lipogenic enzyme genes. J. Biol. Chem. 279, 15662-15669 (2004).
21. J. Cai, X. Jiao, Y. Fang, X. Yu, X. Ding, The orphan nuclear receptor RORa is a potential endogenous protector in renal ischemia/reperfusion injury. FASEB J. Off. Publ. Fed. Am. Soc. Exp. Biol. 33, 5704-5715 (2019).
22. J. Huang, M. Arsenault, M. Kann, C. Lopez-Mendez, M. Saleh, D. Wadowska, M. Taglienti, J. Ho, Y. Miao, D. Sims, J. Spears, A. Lopez, G. Wright, S. Hartwig, The transcription factor Sry-related HMG box-4 (SOX4) is required for normal renal development in vivo. Dev. Dyn. Off. Publ. Am. Assoc. Anat. 242, 790-799 (2013).
23. S. Pak, W. Kim, Y. Kim, C. Song, H. Ahn, Dihydrotestosterone promotes kidney cancer cell proliferation by activating the STAT5 pathway via androgen and glucocorticoid receptors. J. Cancer Res. Clin. Oncol. 145, 2293-2301 (2019).
24. M. Fragiadaki, M. Lannoy, M. Themanns, B. Maurer, W. N. Leonhard, D. J. M. Peters, R. Moriggl, A. C. M. Ong, STAT5 drives abnormal proliferation in autosomal dominant polycystic kidney disease. Kidney Int. 91, 575-586 (2017).
25. N. Miyashita, T. Enokido, M. Horie, K. Fukuda, H. Urushiyama, C. Strell, H. Brunnstrom, P. Micke, A. Saito, T. Nagase, TGF-β-mediated epithelial-mesenchymal transition and tumor-promoting effects in CMT64 cells are reflected in the transcriptomic signature of human lung adenocarcinoma. Sci. Rep. 11, 22380 (2021).
26. M. Diepenbruck, L. Waldmeier, R. Ivanek, P. Berninger, P. Arnold, E. van Nimwegen, G. Christofori, Tead2 expression levels control the subcellular distribution of Yap and Taz, zyxin expression and epithelial-mesenchymal transition. J. Cell Sci. 127, 1523-1536 (2014).
27. C. Contursi, I. M. Wang, L. Gabriele, M. Gadina, J. O'Shea, H. C. 3rd Morse, K. Ozato, IFN consensus sequence binding protein potentiates STAT1-dependent activation of IFNgamma-responsive promoters in macrophages. Proc. Natl. Acad. Sci. U.S.A 97, 91-96 (2000).
28. J. Liu, X. Li, J. Yang, D. Zhang, LncRNA ENSMUST_147219 mediates the progression of ischemic acute kidney injury by targeting the miR-221-5p/IRF6 axis. Apoptosis Int. J. Program. Cell Death (2022).
29. R. Jin, A. Zhao, S. Han, D. Zhang, H. Sun, M. Li, D. Su, X. Liang, The interaction of S100A16 and GRP78 actives endoplasmic reticulum stress-mediated through the IRE1α/XBP1 pathway in renal tubulointerstitial fibrosis. Cell Death Dis. 12, 942 (2021).
30. W. Xu, H. Zhang, Q. Zhang, J. Xu, β-Amyrin ameliorates diabetic nephropathy in mice and regulates the miR-181b-5p/HMGB2 axis in high glucose-stimulated HK-2 cells. Environ. Toxicol. 37, 637-649 (2022).
31. R. Bechara, N. Amatva, R. D. Bailey, Y. Li, F. E. Y. Aggor, D.-D. Li, C. V. Jawale, B. M. Coleman, N. Dai, N. S. Gokhale, T. C. Taylor, S. M. Homer, A. C. Poholek, A. Bansal, P. S. Biswas, S. L. Gaffen, The m(6)A reader IMP2 directs autoimmune inflammation through an IL-17- and TNFα-dependent C/EBP transcription factor axis. Sci. Immunol. 6 (2021).
32. E. Y. Su, A. Spangler, Q. Bian, J. Y. Kasamoto, P. Cahan, Reconstruction of dynamic regulatory networks reveals signaling-induced topology changes associated with germ layer specification. Stem Cell Rep. 17, 427-442 (2022).
33. C. Hinze, C. Kocks, J. Leiz, N. Karaiskos, A. Boltengagen, S. Cao, C. M. Skopnik, J. Klocke, J.-H. Hardenberg, H. Stockmann, I Gotthardt, B. Obermayer, L. Haghverdi, E. Wyler, M. Landthaler, S. Bachmann, A. C. Hocke, V. Corman, J. Busch, W. Schneider, N. Himmerkus, M. Bleich, K.-U. Eckardt, P. Enghard, N. Rajewsky, K. M. Schmidt-Ott, Single-cell transcriptomics reveals common epithelial response patterns in human acute kidney injury. Genome Med. 14, 103 (2022).
34. S. G. Mansour, G. Verma, R. W. Pata, T. G. Martin, M. A. Perazella, C. R. Parikh, Kidney Injury and Repair Biomarkers in Marathon Runners. Am. J. Kidney Dis. Off. J. Natl. Kidney Found. 70, 252-261 (2017).
35. J. Puthumana, H. Thiessen-Philbrook, L. Xu, S. G. Coca, A. X. Garg, J. Himmelfarb, P. K. Bhatraju, T. A. Ikizler, E. Siew, L. B. Ware, K. D. Liu, A. S. Go, J. S. Kaufman, P. L. Kimmel, V. M. Chinchilli, L. Cantley, C. R. Parikh, Biomarkers of inflammation and repair in kidney disease progression. J. Clin. Invest. (2020).
36. M. S. Balzer, T. Doke, Y.-W. Yang, D. L. Aldridge, H. Hu, H. Mai, D. Mukhi, Z. Ma, R. Shrestha, M. B. Palmer, C. A. Hunter, K. Susztak, Single-cell analysis highlights differences in druggable pathways underlying adaptive or fibrotic kidney regeneration. Nat. Commun. 13, 4018 (2022).
37. Y.-A. Lu, C.-T. Liao, R. Raybould, B. Talabani, I. Grigorieva, B. Szomolay, T. Bowen, R. Andrews, P. R. Taylor, D. Fraser, Single-Nucleus RNA Sequencing Identifies New Classes of Proximal Tubular Epithelial Cells in Kidney Fibrosis. J. Am. Soc. Nephrol. JASN 32, 2501-2516 (2021).
38. M. A. Venkatachalam, J. M. Weinberg, W. Kriz, A. K. Bidani, Failed Tubule Recovery, AKI-CKD Transition, and Kidney Disease Progression. J. Am. Soc. Nephrol. JASN 26, 1765-1776 (2015).
39. R. Lan, H. Geng, A. J. Polichnowski, P. K. Singha, P. Saikumar, D. G. McEwen, K. A. Griffin, R. Koesters, J. M. Weinberg, A. K. Bidani, W. Kriz, M. A. Venkatachalam, PTEN loss defines a TGF-β-induced tubule phenotype of failed differentiation and JNK signaling during renal fibrosis. Am. J. Physiol. Renal Physiol. 302, F1210-23 (2012).
40. N. Tiwari, V. K. Tiwari, L. Waldmeier, P. J. Balwierz, P. Arnold, M. Pachkov, N. Meyer-Schaller, D. SchUbeler, E. van Nimwegen, G. Christofori, Sox4 is a master regulator of epithelial-mesenchymal transition by controlling Ezh2 expression and epigenetic reprogramming. Cancer Cell 23, 768-783 (2013).
41. L. Yang, T. Y. Besschetnova, C. R. Brooks, J. V Shah, J. V Bonventre, Epithelial cell cycle arrest in G2/M mediates kidney fibrosis after injury. Nat. Med. 16, 535-43, 1p following 143 (2010).
42. L. Xu, J. Guo, D. G. Moledina, L. G. Cantley, Immune-mediated Tubule Atrophy Promotes Acute Kidney Injury to Chronic Kidney Disease Transition. Nat. Commun. 13, 4892 (2022).
43. A. A. Pozdzik, L. Giordano, G. Li, M.-H. Antoine, N. Quellard, J. Godet, E. De Prez, C. Husson, A.-E. Declèves, V. M. Arlt, J.-M. Goujon, I. Brochériou-Spelle, S. R. Ledbetter, N. Caron, J. L. Nortier, Blocking TGF-β Signaling Pathway Preserves Mitochondrial Proteostasis and Reduces Early Activation of PDGFRβ+ Pericytes in Aristolochic Acid Induced Acute Kidney Injury in Wistar Male Rats. PloS One 11, e0157288 (2016).
44. L. He, Q. Wei, J. Liu, M. Yi, Y. Liu, H. Liu, L. Sun, Y. Peng, F. Liu, M. A. Venkatachalam, Z. Dong, AKI on CKD: heightened injury, suppressed repair, and the underlying mechanisms. Kidney Int. 92, 1071-1083 (2017).
45. J. F. Santibanez, J. Krstic, Transforming Growth Factor-Beta and Urokinase Type Plasminogen Interplay in Cancer. Curr. Protein Pept. Sci. 19, 1155-1163 (2018).
46. F. Laumonnier, F. Bonnet-Brilhault, M. Gomot, R. Blanc, A. David, M.-P. Moizard, M. Raynaud, N. Ronce, E. Lemonnier, P. Calvas, B. Laudier, J. Chelly, J.-P. Fryns, H.-H. Ropers, B. C. J. Hamel, C. Andres, C. Barthélémy, C. Moraine, S. Briault, X-linked mental retardation and autism are associated with a mutation in the NLGN4 gene, a member of the neuroligin family. Am. J. Hum. Genet. 74, 552-557 (2004).
47. F. Xu, K. Chang, J. Ma, Y. Qu, H. Xie, B. Dai, H. Gan, H. Zhang, G. Shi, Y. Zhu, Y. Zhu, Y. Shen, D. Ye, The Oncogenic Role of COL23A1 in Clear Cell Renal Cell Carcinoma. Sci. Rep. 7, 9846 (2017).
48. X. Li, H. Kimura, K. Hirota, K. Kasuno, K. Torii, T. Okada, H. Kurooka, Y. Yokota, H. Yoshida, Synergistic effect of hypoxia and TNF-alpha on production of PAI-1 in human proximal renal tubular cells. Kidney Int. 68, 569-583 (2005).
49. S. Nazir, I. Gadi, M. M. Al-Dabet, A. Elwakiel, S. Kohli, S. Ghosh, J. Manoharan, S. Ranjan, F. Bock, R. C. Braun-Dullaeus, C. T. Esmon, T. B. Huber, E. Camerer, C. Dockendorff, J. H. Griffin, B. Isermann, K. Shahzad, Cytoprotective activated protein C averts Nlrp3 inflammasome-induced ischemia-reperfusion injury via mTORC1 inhibition. Blood 130, 2664-2677 (2017).
50. J. Morita, K. Kano, K. Kato, H. Takita, H. Sakagami, Y. Yamamoto, E. Mihara, H. Ueda, T. Sato, H. Tokuyama, H. Arai, H. Asou, J. Takagi, R. Ishitani, H. Nishimasu, O. Nureki, J. Aoki, Structure and biological function of ENPP6, a choline-specific glycerophosphodiester-phosphodiesterase. Sci. Rep. 6, 20995 (2016).
51. S. G. Mansour, T. G. Martin, W. Obeid, R. W. Pata, K. M. Myrick, L. Kukova, Y. Jia, P. Bjornstad, J. M. El-Khoury, C. R. Parikh, The Role ofVolume Regulation and Thermoregulation in AKI during Marathon Running. Clin. J. Am. Soc. Nephrol. CJASN 14, 1297-1305 (2019).
52. Y. Wang, R. Bellomo, Cardiac surgery-associated acute kidney injury: risk factors, pathophysiology and treatment. Nat. Rev. Nephrol. 13, 697-711 (2017).
53. C. R. Parikh, H. Thiessen-Philbrook, A. X. Garg, D. Kadiyala, M. G. Shlipak, J. L. Koyner, C. L. Edelstein, P. Devarajan, U. D. Patel, M. Zappitelli, C. D. Krawczeski, C. S. Passik, S. G. Coca, Performance of kidney injury molecule-1 and liver fatty acid-binding protein and combined biomarkers of AKI after cardiac surgery. Clin. J. Am. Soc. Nephrol. CJASN 8, 1079-1088 (2013).
54. C. Liu, N. Debnath, G. Mosoyan, K. Chauhan, G. Vasquez-Rios, C. Soudant, S. Menez, C. R. Parikh, S. G. Coca, Systematic Review and Meta-Analysis of Plasma and Urine Biomarkers for CKD Outcomes. J. Am. Soc. Nephrol. JASN 33, 1657-1672 (2022).
55. J. H. Greenberg, P. Devarajan, H. R. Thiessen-Philbrook, C. Krawczeski, C. R. Parikh, M. Zappitelli, Kidney injury biomarkers 5 years after AKI due to pediatric cardiac surgery. Pediatr. Nephrol. Berl. Ger. 33, 1069-1077 (2018).
56. A. Gayoso, J. Shor, JonathanShor/DoubletDetection: doubletdetection v4.2 Zenodo (2022).
57. S. J. Fleming, M. D. Chaffin, A. Arduini, A.-D. Akkad, E. Banks, J. C. Marioni, A. A. Philippakis, P. T. Ellinor, M. Babadi, Unsupervised removal of systematic background noise from droplet-based single-cell experiments using CellBender. bioRxiv, 791699 (2022).
58. Y. Hao, S. Hao, E. Andersen-Nissen, W. M. 3rd Mauck, S. Zheng, A. Butler, M. J. Lee, A. J. Wilk, C. Darby, M. Zager, P. Hoffman, M. Stoeckius, E. Papalexi, E. P. Mimitou, J. Jain, A. Srivastava, T. Stuart, L. M. Fleming, B. Yeung, A. J. Rogers, J. M. McElrath, C. A. Blish, R. Gottardo, P. Smibert, R. Satija, Integrated analysis of multimodal single-cell data. Cell 184, 3573-3587.e29 (2021).
59. G. Korotkevich, V. Sukhov, N. Budin, B. Shpak, M. N. Artyomov, A. Sergushichev, Fast gene set enrichment analysis. bioRxiv, 60012 (2021).
60. N. Kumar, B. Mishra, M. Athar, S. Mukhtar, Inference of Gene Regulatory Network from Single-Cell Transcriptomic Data Using pySCENIC. Methods Mol. Biol. Clifton NJ 2328, 171-182 (2021).
61. F. A. Wolf, P. Angerer, F. J. Theis, SCANPY: large-scale single-cell gene expression data analysis. Genome Biol. 19, 15 (2018).
62. S. Aibar, C. B. González-Blas, T. Moerman, V. A. Huynh-Thu, H. Imrichova, G. Hulselmans, F. Rainbow, J.-C. Marine, P. Geurts, J. Aerts, J. van den Oord, Z. K. Atak, J. Wouters, S. Aerts, SCENIC: single-cell regulatory network inference and clustering. Nat. Methods 14, 1083-1086 (2017).
63. S. A. Williams, M. Kivimaki, C. Langenberg, A. D. Hingoran, J. P. Casas, C. Bouchard, C. Jonasson, M. A. Sarzynski, M. J. Shipley, L. Alexander, J. Ash, T. Bauer, J. Chadwick, G. Datta, R. K. DeLisle, Y. Hagar, M. Hinterberg, R. Ostroff, S. Weiss, P. Ganz, N. J. Wareham, Plasma protein patterns as comprehensive indicators of health. Nat. Med. 25, 1851-1857 (2019).
64. Y. Tan, P. Cahan, SingleCellNet: A Computational Tool to Classify Single Cell RNA-Seq Data Across Platforms and Across Species. Cell Syst. 9, 207-213.e2 (2019).

TABLE 1
Clinical characteristics in patients from the TRIBE-AKI adult
cohort with and without AKI after cardiac surgery

| Characteristics | No AKI (N = 275) | AKI* (N = 47) | P Value |
|---|---|---|---|
| Age (y) | 74 (66-78) | 72 (61.5-76) | 0.05 |
| Male sex | 195 (70.9%) | 36 (76.6%) | 0.42 |
| Race | | | 0.95 |
| White | 258 (93.8%) | 45 (95.7%) | |
| Black | 7 (2.5%) | 1 (2.1%) | |
| Others and unknown | 10 (3.6%) | 1 (2.1%) | |
| Baseline eGFR | 68.6 (54.8-83.1) | 66 (47.6- | 0.13 |
| (mL/min/1.73 m²) | | 79.7) | |
| Baseline ACR (mg/g) | 14 (7-58) | 21 (9-70) | 0.17 |
| Hypertension | 214 (77.8%) | 39 (83.0%) | 0.54 |
| Diabetes mellitus | 105 (38.2%) | 23 (48.9%) | 0.33 |
| Congestive heart failure | 58 (21.1%) | 17 (36.2%) | 0.06 |
| Myocardial infarction | 67 (25.2%) | 14 (29.8%) | 0.51 |

*AKI is defined as severe (KDIGO stage 2-3) AKI.
Continuous variables are presented as median (IQR), categorical variables are presented as N (%)
Abbreviations: ACR, albumin-creatinine ratio; AKI, acute kidney injury; eGFR: estimated glomerular filtration rate.

TABLE 2
Protein fold change in the TRIBE-AKI
adult cohort and 2 independent validation cohorts

| | | | Pediatric Cardiac | | Marathon | |
|---|---|---|---|---|---|---|
| | TRIBE-AKI Adult | | Surgery (N = 68) | | (N = 39) | |
| | (N = 322) | | Fold | | Fold | |
| Protein Name | Fold Change | P Value | Change | P Value | Change | P Value |
| NLGN4X.5357.60 | 1.55 | 1.68E−49 | 1.51 | 4.58E−11 | 1.13 | 1.01E−07 |
| COL23A1.4543.65 | 1.51 | 1.16E−32 | 1.45 | 5.35E−08 | 1.08 | 6.00E−01 |
| TGFB2.4156.74 | 1.73 | 1.66E−47 | 1.44 | 8.68E−09 | 1.34 | 5.90E−08 |
| CD200.5112.73 | 1.2 | 3.54E−37 | 1.09 | 6.67E−06 | 1.22 | 1.09E−11 |
| ENPP6.15579.26 | 0.81 | 9.15E−53 | 0.78 | 6.56E−11 | 1.09 | 3.01E−05 |
| PLG.3710.49 | 0.77 | 3.14E−49 | 0.88 | 1.83E−06 | 0.93 | 3.02E−04 |
| PROC.2961.1 | 0.97 | 1.3E−04 | 0.96 | 1.59E−02 | 0.87 | 1.35E−09 |
| P4HA2.11348.132 | 0.95 | 2.19E−12 | 0.95 | 2.09E−04 | 0.93 | 1.12E−03 |
| AFM.4763.31 | 0.97 | 1.31E−03 | 1.08 | 1.11E−07 | 0.98 | 1.20E−04 |

Fold change is based on comparison of postoperative vs. preoperative levels for the TRIBE-AKI adult cohort and pediatric cardiac surgery cohort and postrace vs. prerace levels in the marathon cohort using paired Wilcoxon tests.
Abbreviations: AFM, afamin; COL23A1, collagen type XXIII α 1 chain; ENPP6, ectonucleotide pyrophosphatase/phosphodiesterase 6; NLGN4X, neuregulin-4 X linked; P4HA2, prolyl 4-hydroxylase; PROC, protein C; PLG, plasminogen; PT, proximal tubule; TGFB2, transforming growth factor β-2.

TABLE 3
Associations between postoperative biomarkers of PT maladaptation,
PT cells at healthy states, and severe AKI after cardiac
surgery in patients from the TRIBE-AKI adult cohort.

| OR (95% CI) | Model 1* | Model 2 | Model 3 | Model 4 | Model 5 |
|---|---|---|---|---|---|
| NLGN4X.5357.60 | 1.87 | 2.09 | 2.48 | 2.65 | 6.29 |
| | (1.09-3.48) | (1.18-4.07) | (1.35-5.07) | (1.37-6.1) | (2.04-20.95) |
| COL23A1.4543.65 | 2.04 | 2.23 | 2.85 | 2.35 | 2.54 |
| | (1.01-4.24) | (1.08-4.74) | (1.32-6.45) | (1.08-5.38) | (1.13-6.01) |
| TGFB2.4156.74 | 2.27 | 2.53 | 3.25 | 2.4 | 2.35 |
| | (1.14-4.66) | (1.24-5.34) | (1.5-7.4) | (1.07-5.61) | (1.03-5.63) |
| CD200.5112.73 | 1.94 | 2.18 | 2.42 | 1.63 | 3.2 |
| | (0.78-5) | (0.85-5.62) | (0.92-6.56) | (0.54-4.46) | (0.77-13.28) |
| ENPP6.15579.26 | 0.29 | 0.18 | 0.21 | 0.29 | 0.04 |
| | (0.05-1.09) | (0.03-0.89) | (0.03-0.91) | (0.04-1.09) | (0.003-0.39) |
| PLG.3710.49 | 0.13 | 0.09 | 0.12 | 0.14 | 0.07 |
| | (0.04-0.44) | (0.02-0.31) | (0.03-0.44) | (0.03-0.54) | (0.01-0.44) |
| PROC.2961.1 | 0.23 | 0.15 | 0.24 | 0.23 | 0.17 |
| | (0.07-0.69) | (0.04-0.49) | (0.06-0.88) | (0.06-0.89) | (0.03-0.83) |
| P4HA2.11348.132 | 0.14 | 0.11 | 0.18 | 0.17 | 0.25 |
| | (0.03-0.67) | (0.02-0.57) | (0.03-0.87) | (0.03-0.84) | (0.02-2.35) |
| AFM.4763.31 | 0.43 | 0.42 | 0.61 | 0.6 | 0.47 |
| | (0.19-1.01) | (0.18-0.97) | (0.25-1.52) | (0.23-1.64) | (0.06-3.59) |

Abbreviations: ACR, albumin-creatinine ratio; AFM, afamin; COL23A1, collagen type XXIII α 1 chain; eGFR: estimated glomerular filtration rate; ENPP6, ectonucleotide pyrophosphatase/phosphodiesterase 6; NLGN4X, neuregulin-4 X linked; OR, odds ratio; P4HA2, prolyl 4-hydroxylase; PROC, protein C; PLG, plasminogen; PT, proximal tubule; TGFB2, transforming growth factor β-2.
*Covariates were sequentially added to the regression models. Model 1 comprised postoperative biomarkers alone; Model 2 additionally adjusted for age, sex, and race; Model 3 additionally adjusted for hypertension, diabetes mellitus, congestive heart failure, myocardial infarction, and baseline eGFR; Model 4 additionally adjusted for baseline ACR; Model 5 additionally adjusted for preoperative biomarker values.
All proteins were presented by names of aptamers used in the SOMAscan assay, and measurements were log2-transformed so that the odds ratio represents an increase in the odds of doubling protein levels.

TABLE 4
Protein concentrations in the TRIBE-AKI adult cohort and in 2 independent validation
cohorts

| | TRIBE-AKI Adult | | Pediatric Cardiac | | | |
|---|---|---|---|---|---|---|
| Protein | (N = 322) | | Surgery (N = 68) | | Marathon (N = 39) | |
| Name* | Preoperative | Postoperative | Preoperative | Postoperative | Prerace | Postrace |
| NLGN4X.5357.60 | 358 (334-391) | 567 (482-680) | 386 (356-470) | 625 (538-837) | 354 (335- | 404 (386-434) |
| | | | | | 385) | |
| COL23A1.4543.65 | 703 (512- | 120 (918- | 556 (500-684) | 849 (728- | 264 (249- | 377 (334-411) |
| | 1,027) | 1,459) | | 1,043) | 285) | |
| TGFB2.4156.74 | 298 (261-380) | 558 (439-718) | 280 (255-321) | 430 (358-518) | 960 (836- | 1,018 (881-1,258) |
| | | | | | 1,170) | |
| CD200.5112.73 | 1,662 (1,532- | 2,096 (1,836- | 1,744 (1,665- | 1,918 (1,824- | 1,521 | 1,867 (1,751- |
| | 1,872) | 2,378) | 1,891) | 2,086) | (1,470- | 2,031) |
| | | | | | 1,597) | |
| ENPP6.15579.26 | 654 (616-719) | 543 (499-590) | 735 (680-810) | 567 (525-618) | 745 (683- | 800 (725-857) |
| | | | | | 768) | |
| PLG.3710.49 | 23,389 (20,593- | 18,045 | 20,384 | 17,725 | 23,013 | 21,165 (19,355- |
| | 25,869) | (15,981- | (18,367- | (16,018- | (20,305- | 25,348) |
| | | 20,348) | 23,113) | 19,724) | 26,357) | |
| PROC.2961.1 | 73,950 (63,166- | 68,505 | 65,910 | 58,245 | 76,172 | 64,882 (58,153- |
| | 83,586) | (61,261- | (54,156- | (52,598- | (71,758- | 72,615) |
| | | 79,560) | 73,059) | 66,119) | 80,537) | |
| P4HA2.11348.132 | 1,106 (999- | 1,048 (962- | 935 (870- | 868 (814-934) | 1,119 | 1,067 (948- |
| | 1,228) | 1,131) | 1,028) | | (1,043- | 1,204) |
| | | | | | 1,275) | |
| AFM.4763.31 | 45,905 (40,285- | 45,249 | 40,233 | 46,759 | 49,699 | 48,302 (43,428- |
| | 51,472) | (39,436- | (36,576- | (42,098- | (44,046- | 51,216) |
| | | 51,053) | 46,950) | 50,751) | 52,624) | |

*Values are presented as median (IQR) of normalized aptamer measurements in relative fluorescence intensities.
Abbreviations: AFM, afamin; COL23A1, collagen type XXIII α 1 chain; ENPP6, ectonucleotide pyrophosphatase/phosphodiesterase 6; NLGN4X, neuregulin-4 X linked; P4HA2, prolyl 4-hydroxylase; PROC, protein C; PLG, plasminogen; PT, proximal tubule; TGFB2, transforming growth factor β-2.

TABLE 5
Correlation of preoperative candidate biomarkers of PT
maladaptation and PT cells at healthy states with
baseline eGFR in 322 TRIBE-AKI adult participants

| | Spearman | |
|---|---|---|
| Protein Name | Correlation Coefficient | P Value |
| NLGN4X.5357.60 | −0.02 | 0.68 |
| COL23A1.4543.65 | 0.03 | 0.61 |
| TGFB2.4156.74 | 0.07 | 0.21 |
| CD200.5112.73 | −0.01 | 0.85 |
| ENPP6.15579.26 | 0.06 | 0.3 |
| PLG.3710.49 | 0.24 | <0.001 |
| PROC.2961.1 | 0.3 | <0.001 |
| P4HA2.11348.132 | 0.17 | 0.003 |
| AFM.4763.31 | 0.27 | <0.001 |

Abbreviations: AFM, afamin; COL23A1, collagen type XXIII α 1 chain; eGFR: estimated glomerular filtration rate; ENPP6, ectonucleotide pyrophosphatase/phosphodiesterase 6; NLGN4X, neuregulin-4 X linked; P4HA2, proly1 4-hydroxylase; PROC, protein C; PLG, plasminogen; PT, proximal tubule; TGFB2, transforming growth factor β-2.

TABLE 6
Changes in urinary biomarkers of PT maladaptation and PT cells at healthy states in 54
TRIBE-AKI participants

| | | | Fold | |
|---|---|---|---|---|
| Protein Name* | Preoperative | Postoperative | Change^{#} | P Value |
| NLGN4X.5357.60 | 19.90 | 52.15 | 2.62 | 2.19E−08 |
| | (15.83, 24.3) | (34.15, 83.8) | | |
| COL23A1.4543.65 | 22.60 | 61.00 | 2.84 | 3.77E−07 |
| | (16.55, 33.05) | (34.93, 111.85) | | |
| TGFB2.4156.74 | 31.80 | 25.60 | 0.82 | 6.79E−02 |
| | (20.7, 48.67) | (17.12, 36.05) | | |
| CD200.5112.73 | 2,213.25 | 1,364.05 | 0.69 | 1.92E−02 |
| | (1,326.02, 2,893.18) | (891.87, 2,186.9) | | |
| PLG.3710.49 | 103.80 | 136.1 | 1.81 | 3.72E−02 |
| | (26.83, 234.72) | (47.15, 356.48) | | |
| PROC.2961.1 | 113.85 | 135.8 | 1.48 | 1.85E−01 |
| | (59.35, 222.97) | (87.17, 305.02) | | |
| AFM.4763.31 | 28,715.05 | 64,010.65 | 1.84 | 1.67E−03 |
| | (14,351.73, 56,946.27) | (23,846.8, | | |
| | | 98,098.25) | | |
| NLGN4X.5357.60/UCr ratio | 0.24 | 0.89 | 3.48 | 1.14E−08 |
| | (0.13, 0.39) | (0.4, 1.8) | | |
| COL23A1.4543.65/UCr ratio | 0.26 | 0.85 | 3.23 | 8.95E−08 |
| | (0.18, 0.44) | (0.53, 1.5) | | |
| TGFB2.4156.74 | 0.36 | 0.38 | 0.82 | 8.3E−01 |
| | (0.26, 0.55) | (0.2, 0.7) | | |
| CD200.5112.73/UCr ratio | 22.78 | 20.5 | 0.92 | 2.93E−01 |
| | (17.39, | (13.84, −28.75) | | |
| | 27.72) | | | |
| PLG.3710.49/UCr ratio | 1.09 | 1.49 | 2.61 | 3.61E−03 |
| | (0.36, 3.04) | (0.67, 7.31) | | |
| PROC.2961.1/UCr ratio | 1.31 | 2.26 | 1.99 | 2.3E−03 |
| | (0.55, 2.52) | (0.96, 4.92) | | |
| AFM.4763.31/UCr ratio | 325.03 | 813.83 | 2.32 | 7.75E−05 |
| | (135.45, 672.37) | (289.24, 2,052.05) | | |

*Values are presented as median (IQR) of normalized aptamer measurements in relative fluorescence intensities with and without normalization by urine creatinine concentration (mg/dL).
^{#}Fold change is based on comparing postoperative versus preoperative protein levels
Abbreviations: AFM, afamin; COL23A1, collagen type XXIII α 1 chain; NLGN4X, neuregulin-4 X linked; PROC, protein C; PLG, plasminogen; PT, proximal tubule; TGFB2, transforming growth factor β-2; UCr, urine creatinine.

TABLE 7
Performance of biomarkers of PT maladaptation and PT cells at
healthy states in predicting severe AKI after cardiac surgery
when added to known kidney disease biomarkers

| Biomarker | AUC | Biomarker | AUC | Biomarker | AUC |
|---|---|---|---|---|---|
| HAVCR1 | 0.73 | LCN2 | 0.75 | PLAUR | 0.74 |
| (KIM1) | (0.64-0.82) | (NGAL) | (0.66-0.84) | (suPAR) | (0.65-0.83) |
| +TGFB2 | 0.76 | +TGFB2 | 0.77 | +TGFB2 | 0.76 |
| | (0.68-0.84) | | (0.69-0.85) | | (0.68-0.85) |
| | | | | | * |
| +COL23A1 | 0.77 | +COL23A1 | 0.77 | +COL23A1 | 0.77 |
| | (0.69-0.84) | | (0.69-0.85) | | (0.69-0.85) |
| | * | | | | * |
| +NLGN4X | 0.78 | +NLGN4X | 0.79 | +NLGN4X | 0.78 |
| | (0.7-0.86) | | (0.7-0.87) * | | (0.7-0.86) |
| | ** | | | | ** |
| +CD200 | 0.75 | +CD200 | 0.77 | +CD200 | 0.76 |
| | (0.67-0.83) | | (0.69-0.85) | | (0.68-0.84) |
| +PLG | 0.77 | +PLG | 0.78 | +PLG | 0.78 |
| | (0.68-0.85) | | (0.69-0.87) | | (0.69-0.86) |
| | * | | * | | * |
| +PROC | 0.76 | +PROC | 0.77 | +PROC | 0.77 |
| | (0.67-0.84) | | (0.68-0.85) | | (0.68-0.85) |
| | * | | | | * |
| +ENPP6 | 0.77 | +ENPP6 | 0.78 | +ENPP6 | 0.77 |
| | (0.69-0.85) | | (0.7-0.86) * | | (0.69-0.85) |
| | * | | | | * |
| +P4HA2 | 0.77 | +P4HA2 | 0.78 | +P4HA2 | 0.77 |
| | (0.68-0.85) | | (0.69-0.86) | | (0.68-0.84) |
| | * | | | | |
| +AFM | 0.75 | +AFM | 0.77 | +AFM | 0.75 |
| | (0.66-0.83) | | (0.68-0.85) | | (0.66-0.83) |

Predictive performance (AUC) was compared using bootstrap analysis of 1,000 samples.
* P < 0.05;
** P < 0.01.

TABLE 8
Correlation in gene expression between markers of PT maladaptation
and PT cells at healthy states and fibrosis markers in
mouse models of kidney atrophy and repair after IRI

| Marker/ | Tgfb2 | | Col23al | | Enpp6 | | Proc | |
|---|---|---|---|---|---|---|---|---|
| Fibrosis | Correlation | P | Correlation | P | Correlation | P | Correlation | P |
| Gene | * | Value | * | Value | * | Value | * | Value |
| Collal | 0.59 | 9.41E− | 0.01 | 0.93 | −0.53 | 2.11E− | −0.60 | 6.95E− |
| | | 07 | | | | 05 | | 07 |
| Col3al | 0.49 | 9.85E− | −0.13 | 0.34 | −0.41 | 1.27E− | −0.47 | 2.04E− |
| | | 05 | | | | 03 | | 04 |
| Fnl | 0.55 | 6.50E− | −0.12 | 0.37 | −0.46 | 3.01E− | −0.54 | 1.19E− |
| | | 06 | | | | 04 | | 05 |
| Pdgfrb | 0.71 | 4.63E− | 0.15 | 0.26 | −0.62 | 1.64E− | −0.50 | 6.94E− |
| | | 10 | | | | 07 | | 05 |
| Acta2 | 0.26 | 0.051 | −0.30 | 0.02 | −0.18 | 0.16 | −0.32 | 0.013 |

* Assessed using Pearson correlation.

1. A method for detecting acute tubular injury (ATI) comprising the steps of:
(a) detecting increased expression of neuregulin-4 X linked (NLGN4X), collagen type XXIII α 1 chain (COL23A1) and transforming growth factor β-2 (TGFB2) relative to a reference in a sample obtained from a patient; and
(b) detecting decreased expression of ectonucleotide pyrophosphatase/phosphodiesterase 6 (ENPP6), plasminogen (PLG) and protein C (PROC) in the patient sample.

2. The method of claim 1, wherein step (a) further comprises detecting expression of CD200.

3. The method of claim 1, wherein step (b) further comprising detecting expression of prolyl 4-hydroxylase (P4HA2) and/or afamin (AFM).

4. The method of claim 1, wherein the acute tubular injury is proximal tubular injury (PTI).

5. The method of claim 1, wherein the sample is plasma or urine.

6. A method for identifying a patient as likely to develop acute kidney injury (AKI) comprising the steps of:
(a) detecting increased expression levels of NLGN4X, COL23A1, and TFGB2 relative to a reference in a sample obtained from the patient; and
(b) detecting decreased expression levels of ENPP6, PLG and PROC relative to a reference in the patient sample, thereby detecting ATI in the patient which is likely to develop into AKI.

7. The method of claim 6, wherein step (a) further comprises detecting expression of CD200.

8. The method of claim 6, wherein step (b) further comprising detecting expression of P4HA2 and/or AFM.

9. The method of claim 6, wherein the ATI is PTI.

10. The method of claim 6, wherein the sample is plasma or urine.

11. The method of claim 6, wherein a patient identified in step (b) is treated for AKI.

12. The method of claim 11, wherein the AKI treatment comprises one or more of renal replacement therapy, angiotensin-converting-enzyme inhibitors, Ang II type I (ATI) blockers, corticosteroids, immunosuppressants, artemisinin and derivatives thereof, drag-reducing polymers, endothelin subtype A receptor antagonists (pro)renin receptor antagonists and 5-HT₃-targeting drugs.

13. (canceled)

14. A method for identifying a patient as having PTI comprising the step of measuring the level of NLGN4X, COL23A1, TGFB2, ENPP6, PLG, PROC and optionally one or more of CD200, P4HA2 and AFM in a biological sample obtained from the patient, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference identifies the patient as having PTI.

15. A method of identifying a cardiac surgery patient as having a high risk of developing severe AKI comprising the step of measuring the level of NLGN4X, COL23A1, TGFB2, ENPP6, PLG, PROC and optionally one or more of CD200, P4HA2 and AFM in a biological sample obtained from the patient, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference identifies the patient as having a high risk of developing severe AKI.

16. A method for detecting drug-induced PTI comprising the step of measuring the level of NLGN4X, COL23A1, TGFB2, ENPP6, PLG, PROC and optionally one or more of CD200, P4HA2 and AFM in a biological sample obtained from the patient, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference identifies the patient as having drug-induced PTI.

17. The method of claim 16, further comprising the step of ceasing administration of the drug.

18. The method of claim 16, wherein the serum creatinine (SCr) level of the patient has not increased relative to a control and a diagnosis of AKI has not been made.

19. A method for monitoring kidney injury in a patient with decompensated cirrhosis comprising the step of measuring the level of NLGN4X, COL23A1, TGFB2, ENPP6, PLG, PROC and optionally one or more of CD200, P4HA2 and AFM in a biological sample obtained from the patient, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference identifies the patient as having PTI, or (c) no change in the amount of one or more of NLGN4X, COL23A1, TGFB2, ENPP6, PLG, PROC and optionally one or more of CD200, P4HA2 and AFM relative to a reference identifies the patient not having PTI.

20. The method of claim 19, wherein a patient identified as having PTI is given a liver or kidney transplantation.

21. The method of claim 19, wherein a patient identified in step (c) is administered volume expansion therapy and/or vasoconstrictive therapy.

22. A method for managing AKI in a patient with decompensated heart failure comprising the step of measuring the level of NLGN4X, COL23A1, TGFB2, ENPP6, PLG, PROC and optionally one or more of CD200, P4HA2 and AFM in a biological sample obtained from the patient, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference identifies the patient as having PTI, or (c) no change in the amount of one or more of NLGN4X, COL23A1, TGFB2, ENPP6, PLG, PROC and optionally one or more of CD200, P4HA2 and AFM relative to a reference identifies the patient not having PTI.

23. The method of claim 22, wherein a patient identified as having PTI is administered diuresis to relieve renal congestion and restore hemodynamics.

24. The method of claim 22, wherein a patient identified in step (c) is treated for AKI.

25. The method of claim 24, wherein the AKI treatment comprises one or more of renal replacement therapy, angiotensin-converting-enzyme inhibitors, Ang II type I (ATI) blockers, corticosteroids, immunosuppressants, artemisinin and derivatives thereof, drag-reducing polymers, endothelin subtype A receptor antagonists (pro)renin receptor antagonists and 5-HT₃-targeting drugs.

26. A method for risk stratification for AKI transitioning to chronic kidney disease (CKD) comprising the step of measuring the level of NLGN4X, COL23A1, TGFB2, ENPP6, PLG, PROC and optionally one or more of CD200, P4HA2 and AFM in a biological sample obtained from the patient, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference identifies the patient as developing incident CKD or experiencing CKD progression after severe AKI.

27. The method of claim 26, further comprising the step of administering to the patient developing incident CKD or experiencing CKD progression after severe AKI one or more of angiotensin converting enzyme inhibitors, angiotensin receptor blockers and sodium glucose cotransporter inhibitors.

28. A method for evaluating nephrotoxicity in a preclinical rodent toxicity study comprising the step of measuring the level of NLGN4X, COL23A1, TGFB2, ENPP6, PLG, PROC and optionally one or more of CD200, P4HA2 and AFM in a biological sample obtained from the rodent who has been administered a drug, wherein (a) an increase in the amount of NLGN4X, COL23A1, TGFB2 and optionally CD200 relative to a reference and (b) a decrease in the level of ENPP6, PLG, PROC and optionally P4HA2 and/or AFM relative to a reference indicates early injury to the proximal tubules and significant nephrotoxicity.

29. The method of claim 15, wherein the biological sample comprises blood or urine.
